(19) United States
(12) Patent Application Publication
(10) Pub. No.: US 2023/0381373 A1
(43) Pub. Date: Nov. 30, 2023

(54) Rapidly synthesized extracellular matrix-based gels and patterning techniques, from micro- to macro-scale

(71) Applicant: Yale University, New Haven, CT (US)

(72) Inventors: Michael Mak, New Haven, CT (US); Xiangyu Gong, New Haven, CT (US)

(21) Appl. No.: 18/317,526

(22) Filed: May 15, 2023

Related U.S. Application Data
(60) Provisional application No. 63/341,689, filed on May 13, 2022.

(51) Int. Cl. A61L 27/24; A61L 27/26; A61L 27/50

(52) U.S. Cl. CPC A61L 27/24; A61L 27/26; A61L 27/507; A61L 2430/20; A61L 2430/40

(57) ABSTRACT

In some aspects, the present invention provides a method of forming crowded collagen based constructs, having the steps of providing a collagen solution in a buffer, providing a macromolecular bath solution, and delivering the collagen solution into the macromolecular bath solution to form crowded collagen constructs. In other aspects, the present invention provides a method of forming crowded cellular based constructs, having the steps of providing a cellular solution in a buffer, providing a macromolecular bath solution, and delivering the cellular solution into the macromolecular bath solution to form crowded cellular constructs.

### CROSS REFERENCE TO RELATED APPLICATIONS

This application claims priority to U.S. Provisional Patent Application No. 63/341,689, filed May 13, 2022 incorporated herein by reference in its entirety.

### STATEMENT REGARDING FEDERALLY SPONSORED RESEARCH OR DEVELOPMENT

This invention was made with government support under Grant No. R35GM142875 awarded by the National Institutes of Health. The government has certain rights in the invention.

### BACKGROUND OF THE INVENTION

Epithelial organoids are stem cell-derived tissues that approximate aspects of real organs, and thus, they have potential as powerful tools in basic and translational research. By definition, they self-organize, but the structures formed are often heterogeneous and irreproducible, which limits their use in the lab and clinic (Gjoresvski, N. et al., 2022, Science 375(6576):eaaw9021). The approaches that are used at present to derive these organoids in three-dimensional matrices result in stochastically developing tissues with a closed, cystic architecture that restricts lifespan and size, limits experimental manipulation, and prohibits homeostasis (Nikolaev, M. et al, 2020, Nature 585:574-578). Further, organoids have largely not addressed longer length-scale (>0.5 mm) tissue developmental processes beyond local self-organization.

Thus, there is a pressing need in the art for the development of a method for generating rapidly synthesized extracellular matrix (ECM)-based scaffolds of various shapes and sizes, spanning micro to macroscales. The present invention meets this need.

### SUMMARY OF THE INVENTION

In some aspects, the present invention provides a method of forming crowded collagen based constructs, having the steps of providing a collagen solution in a buffer, providing a macromolecular bath solution, and delivering the collagen solution into the macromolecular bath solution to form crowded collagen constructs.

In some embodiments, the macromolecular bath solution has a high molecular weight polyethylene glycol (PEG), wherein the molecular weight of the PEG is ranging between 0-30000 Da. In some embodiments, the molecular weight of the PEG is ranging between 0-2000 Da. In some embodiments, the molecular weight of the PEG is 8000 Da. In some embodiments, the concentration of the PEG in the macromolecular bath solution is ranging between 0-1000 mg/mL.

In some embodiments, the buffer is selected from the group consisting of: NaOH, phosphate buffered saline (PBS), cell culture media, acetic acid, hydrochloric acid, pepsin and water. In some embodiments, the collagen solution comprises collagen type I.

In some embodiments, the collagen solution has a collagen concentration ranging between 0-30 mg/mL. In some embodiments, the collagen solution has a collagen concentration ranging between 2-6 mg/mL. In some embodiments, the collagen solution has a collagen concentration ranging between 0-2 mg/mL. In some embodiments, the collagen solution has a collagen concentration higher than 30 mg/mL.

In some embodiments, the method further has the step of adding one or more cells to the collagen solution. In some embodiments, the one or more cells are selected from the group consisting of: endothelial cells, stem cells, induced pluripotent stem cells, cancer cells, stem cell-derived cells, stromal cells, fibroblasts, immune cells, somatic cells, blood cells, and reproductive cells.

In some embodiments, the method further has the step of adding one or more materials to the collagen solution. In some embodiments, the one or more materials are selected from the group consisting of: extracellular matrix proteins, proteoglycans, fibrinogen, thrombin, fibrin, collagen IV, laminin, basement membrane proteins, Matrigel, and Geltrex.

In some embodiments, the method further has the step of adding one or more materials to the macromolecular bath solution. In some embodiments, the one or more materials are selected from the group consisting of: agarose, gelatin, methylcellulose, and viscosity-modifying reagent.

In some embodiments, the step of delivering the collagen solution into the macromolecular bath solution includes using a pipette to extrude the collagen solution into the macromolecular bath solution.

In some embodiments, the step of delivering the collagen solution into the macromolecular bath solution includes using a syringe to extrude the collagen solution into the macromolecular bath solution.

In some embodiments, the step of delivering the collagen solution into the macromolecular bath solution includes aerosolizing the collagen solution and spraying the collagen solution into the macromolecular bath solution.

In some embodiments, the method further has the steps of providing a mold, filling the mold with the macromolecular bath solution, depositing the collagen solution into the mold. In some embodiments, the step of depositing the collagen solution into the mold comprises flowing the collagen solution into the mold.

In some aspects, the present invention relates to a crowded collagen construct formed by any disclosed method. In some embodiments, the construct is formed in the shape of a disk. In some embodiments, the construct is formed in an elongated strip. In some embodiments, the construct is formed into a vascularized tissue construct.

In some embodiments, the construct is formed into fiber-like bundles. In some embodiments, the fiber-like bundles have diameters ranging from 0-1000 μm. In some embodiments, the fiber-like bundles have diameters greater than 1000 μm.

In some embodiments, the fiber-like bundles are formed into a porous scaffold. In some embodiments, the pore sizes of the scaffold range between 1-1000 μm. In some embodiments, the fiber-like bundles are formed into organoids. In some embodiments, the fiber-like bundles are formed into tumor spheroids.

In some embodiments, any disclosed method further comprises differentiating the construct in situ, culturing the construct, maturing the construct, biochemically stimulating the construct, biophysically stimulating the construct, biologically stimulating the construct, biochemically modifying the construct, biophysically modifying the construct, and biologically modifying the construct.

In some aspects, the present invention provides a method of forming crowded cellular based constructs, having the steps of providing a cellular solution in a buffer, providing a macromolecular bath solution, and delivering the cellular solution into the macromolecular bath solution to form crowded cellular constructs.

In some embodiments, the macromolecular bath solution comprises a high molecular weight polyethylene glycol (PEG), wherein the molecular weight of the PEG is ranging between 0-30000 Da. In some embodiments, the concentration of the PEG in the macromolecular bath solution is ranging between 0-1000 mg/mL. In some embodiments, the buffer is selected from the group consisting of: NaOH, phosphate buffered saline (PBS), cell culture media, acetic acid, hydrochloric acid, pepsin and water.

In some embodiments, the method further includes adding one or more cells to the cellular solution, wherein the one or more cells are selected from the group consisting of: parenchymal cells, collenchyma cells, sclerenchyma cells, xylem cells, phloem cells, meristematic cells, epidermal cells, chloroplasts, plastids, leucoplasts, chromoplasts, plasmodesma, amyloplast, guard cells, vessel element cells, and sieve tube element cells.

In some embodiments, the method further includes adding one or more materials to the macromolecular bath solution; wherein the one or more materials are selected from the group consisting of: agarose, gelatin, slurry material, sacrificial slurry material, methylcellulose, and viscosity-modifying reagent. In some embodiments, the step of delivering the cellular solution into the macromolecular bath solution includes using any of a pipette, a syringe, a nozzle, to extrude the cellular solution into the macromolecular bath solution.

In some embodiments, the method further includes providing a mold, filling the mold with the macromolecular bath solution, depositing the cellular solution into the mold. In some embodiments, the step of depositing the cellular solution into the mold comprises flowing the cellular solution into the mold. In some embodiments, the step of delivering the cellular solution into the macromolecular bath solution includes aerosolizing the cellular solution and spraying the cellular solution into the macromolecular bath solution.

In some embodiments, the crowded cellular construct formed by any disclosed method includes at least one of a disk, an elongated strip, a vascularized tissue construct, a microvascular network construct, a cell-laden construct, a patterned construct, and a fiber-like bundle.

### BRIEF DESCRIPTION OF THE DRAWINGS

The following detailed description of invention will be better understood when read in conjunction with the appended drawings. It should be understood, however, that the invention is not limited to the precise arrangements and instrumentalities of the embodiments shown in the drawings.

FIG. 1A is a flowchart depicting an exemplary method of forming crowded collagen constructs. FIG. 1B is a flowchart depicting an exemplary method of forming crowded cellular constructs.

FIG. 2A through FIG. 2F depict rapid collagen shaping/fabrication using macromolecular crowding effect. FIG. 2A depicts fabrication of μ-bundles. FIG. 2B depicts fabrication of mesoscale strips. FIG. 2C depicts fabrication of mesoscale strips.

FIG. 2D depicts fabrication of mesoscale disks. FIG. 2E depicts instant fabrication of collagen p-particles. FIG. 2F depicts rapid free hand printing.

FIG. 3 depicts the principle of crowding effects on collagen. Collagen solution droplets are rapidly solidified by PEG8000 solution to adopt a defined disk shape. Shape is locked in within seconds and fully formed in 3 minutes. In contrast, a regular buffer without PEG does not have the rapid gelation effects on the collagen solution.

FIG. 4 depicts PEG8000 crowding effects on collagen at varied concentrations.

FIG. 5 depicts that crowded disk area and diameter can be controlled by the volume of the collagen solution.

FIG. 6 depicts methods to create microscale collagen bundles with different concentration by crowding effects. The fluorescence intensity is decreasing with the collagen concentration decreasing. The morphology showing a gradient from sharp bend to smoother. Further quantifying: average diameter; density.

FIG. 7 depicts how microscale collagen bundles can be created by both neutralized collagen solution or acidified solution.

FIG. 8 depicts methods to create collagen microbeads.

FIG. 9 depicts methods to create collagen strip by crowding effects in a high throughput manner. Physiological relevant due to the strip morphology.

FIG. 10 depicts crowding induced rapid shaping of collagen with a dense shell due to rapid gelation.

FIG. 11 depicts guided growth of microtissues by a collagen μ-bundle scaffold. Increases surface area for cell growth. Application 1: “fibrotic spheroids” mimicking liver fibrosis in patients, high throughput screening taking into account cues from tissue-relevant fibrosis; Application 2: guided formation of vasculature networks.

FIG. 12 depicts bundle scaffolded liver multicellular spheroids termed as “fibrotic spheroids,” with increases spheroid size and unique cell-collagen interactions.

FIG. 13 depicts a proliferation assay on the liver fibrotic spheroids.

FIG. 14 depicts methods to create collagen based microvasculature. Microscale bundle guided microvasculature formation is shown.

FIG. 15 depicts methods to create collagen based microvasculature. Microscale bundle guided microvasculature formation is shown. VE-Cad as a marker for the cell junction formation.

FIG. 16 depicts that the bundle promotes HUVEC vascular formation and cell viability on low adhesion plates.

FIG. 17 depicts crowded bundle incorporated strips based micro-vessels. HUVECs can form interconnected tubular network in strips with bundles. Crowded bundles can guide network formation, and connected between an outer surface and an inner region.

FIG. 18 depicts crowded strip based micro-vessels showing immunofluorescence staining of the strips. Shown in red is VE-Cad of the interconnected tubular network. In some embodiments, HUVECs can form a monolayer surface in the control strip. Also shown is VE-Cad of the internal network (strip with bundles); and the live/dead or apoptosis assay.

FIG. 19 depicts fabrication of a 3D macroscopic strip like tissue comprising liver cells that show high cell viability (>95%).

FIG. 20 depicts crowding facilitated fabrication method for strips with tissue relevant cell density cultured in suspension, iPSC. Crowding-induced rapid collagen gelation renders uniform cell distribution by preventing cells from sinking.

FIG. 21 depicts that there is no significant differences between collagen/Matrigel and collagen strip when embedded with iPSCs.

FIG. 22 depicts iPSC based crowded strips. iPSCs can maintain pluripotency in the strip.

FIG. 23 depicts that iPSC spheroids (traditional method) do not have relevant geometry for further differentiation to functional intestinal tissues.

FIG. 24 depicts a crowded strip based definitive endoderm organoid. iPSCs can be differentiated to definitive endoderm (DE, labeled by SOX17 and FOXA2) directly in the strip. Differentiation begins at day 1 after iPSC were embedded in Matrigel and the differentiation lasts for 3 days.

FIG. 25 depicts tissue folding and module manufacture based on crowded collagen. Dual cell constructs are shown where HUVEC disks or strips merged with normal human lung fibroblast (NHLF) disks or strips.

FIG. 26 depicts that HUVECs self-assemble on collagen strips in suspension with or without the addition of 75 ng/mL VEGF.

FIG. 27 depicts that VEGF promotes inner lumen growth and disrupts surface HUVEC organization and stability.

FIG. 28 depicts iPSC strips.

FIG. 29 depicts iPSC strips with internal luminal cellular organizations and high pluripotency.

FIG. 30 depicts inducing iPSC strips into germ layers: embryoid body (EB) and definitive endoderm (DE).

FIG. 31 depicts inducing DE strip to mid-hind gut organoids.

FIG. 32 depicts inducing DE strip to intestinal organoids.

FIG. 33 depicts the full protocol for the in situ differentiation of iPSC strips into macroscopic intestinal tubes. Expressions of DE, mid-hind gut, and intestine markers and tissue morphology at different stages are evaluated.

FIG. 34 depicts in situ differentiation of iPSC strips into macroscopic intestinal tubes up to 5 weeks. Expressions of intestine markers and tissue morphology are evaluated.

FIG. 35 depicts intestinal tubes directly derived from collagen strips packed with mouse intestinal cells.

FIG. 36 depicts inducing EB strip to neuronal organoids.

FIG. 37 depicts suboptimal expansion in a free floating culture (no Matrigel encapsulation).

FIG. 38 depicts crowded collagen strips created by free hand drawing.

FIG. 39 depicts tissue folding and module manufacture based on crowded collagen. MSC-crowded collagen disk tissue folding.

FIG. 40 depicts folding of collagen disks by osteogenic MSCs.

FIG. 41 depicts tissue folding at high cell density due to cell contraction on the dense shell.

### DETAILED DESCRIPTION

It is to be understood that the figures and descriptions of the present invention have been simplified to illustrate elements that are relevant for a clear understanding of the present invention, while eliminating, for the purpose of clarity many other elements found in the field of tissue engineering. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, 1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

Rapidly Synthesized Extracellular Matrix-Based Gels and Patterning Techniques. From Micro- to Macro-Scale

The present invention provides a method for forming crowded cellular constructs, and articles, such as engineered tissue, and for example crowded collagen constructs. In one embodiment, the method of the present invention is able to rapidly generate scaffolds (within seconds) to enable fast synthesis of tissues from the ground-up, without requiring time for incubation. In one embodiment, the method of the present invention facilitates instant positioning of cells in a 3D environment. In one embodiment, the method of the present invention may be used in bioprinting. In one embodiment, the method of the present invention is able to generate various geometries, including but not limited to long, macroscopic tissue strips. In one embodiment, the method of the present invention is able to generate large-scaled tissue constructs from the ground-up. In one embodiment, the method of the present invention enables a novel means for rapid bioprinting using natural Extracellular matrix (ECM) proteins. In one embodiment, the method of the present invention allows fabrication of collagen constructs in any shape including but not limited to microbundles, mesoscale strips, mesoscale disks, microparticles, etc.

In some embodiments, the crowded cellular constructs may comprise plant-based cells. In some embodiments, the crowded cellular constructs may comprise animal-based cells. In some embodiments, the crowded cellular constructs may comprise microbial cells. While the application primarily discusses collagen constructs and/or structures, it should be appreciated that any plant or animal cell-type may be used with the disclosed methods. Any methods and/or descriptions as they may be applied to collagen constructs may also be applied to any plant-based or animal-based cellular constructs.

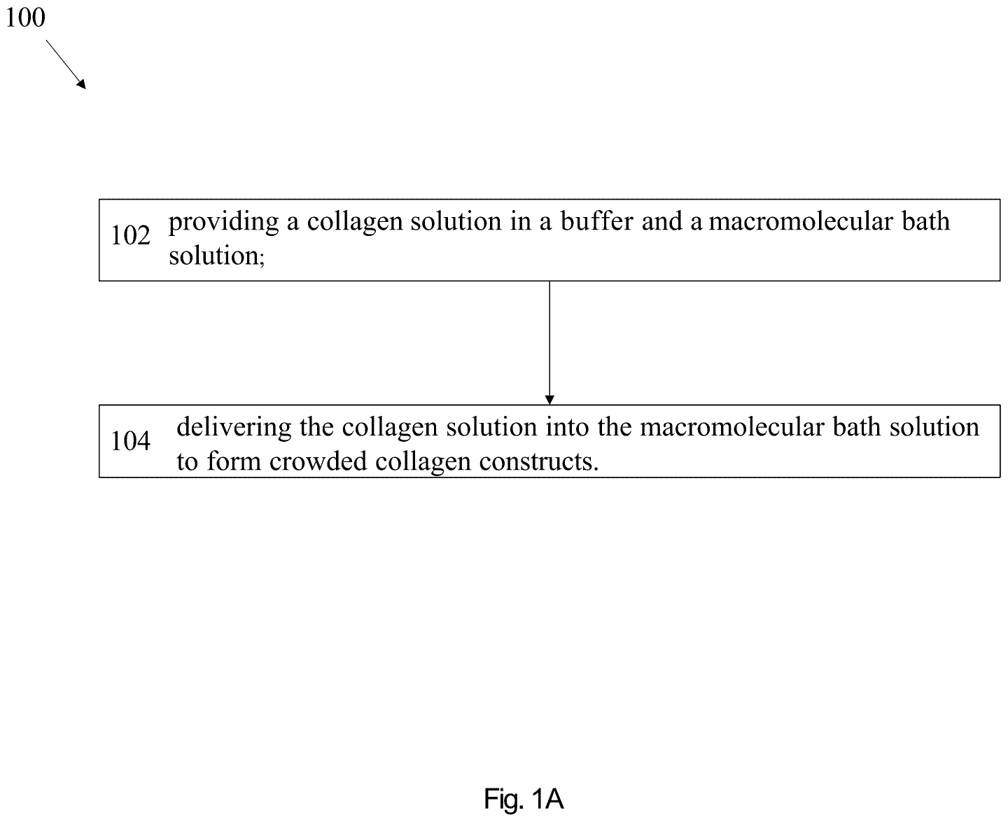

Referring now to FIG. 1, an exemplary method of forming crowded cellular (e.g. collagen based) constructs is depicted. Method 100 begins with step 102, wherein a cellular solution in a buffer and a macromolecular bath solution is provided. It should be noted that “crowding solution”, “crowding bath”, “macromolecular bath”, and “support bath” may be used interchangeably herein. In one embodiment, the macromolecular bath solution may comprise any material including but not limited to polyethylene glycol (PEG), Dextran, a water-soluble polymer, Glycosaminoglycans, Hyaluronan Acid, or any other suitable solution that contains large molecules. In one embodiment, the macromolecular bath solution may comprise PEG. The PEG may have a number average molecular weight (Mn) in a range from about 0 to about 30,000 Da. In one embodiment, the PEG may have a high molecular weight of about 8000 Da. In one embodiment, the macromolecular bath solution may have any suitable concentration. For example, from about 10% (w/v) to about 50% (w/v) in water or phosphate buffered saline (PBS), or from about 20% (w/v) to about 40% (w/v). In one embodiment, the macromolecular bath solution may be prepared by dissolving high molecular weight (˜8000 Da) polyethylene glycol PEG8000 (Sigma) in 1×PBS at the concentration of 200 mg/mL.

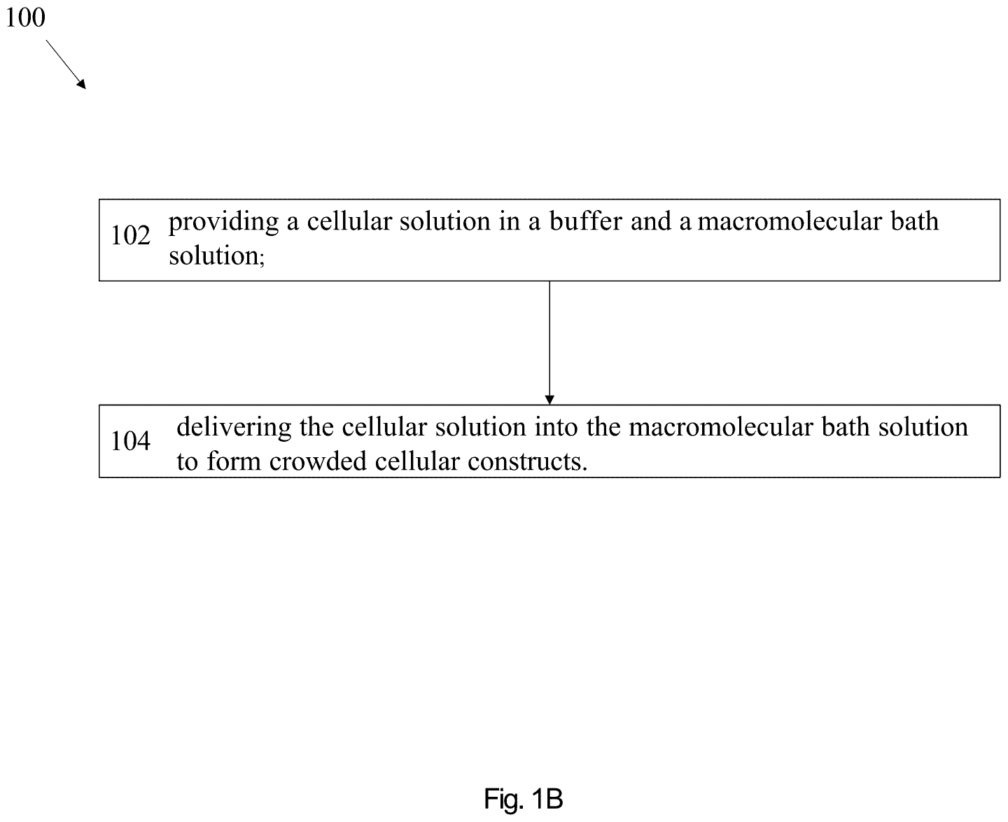

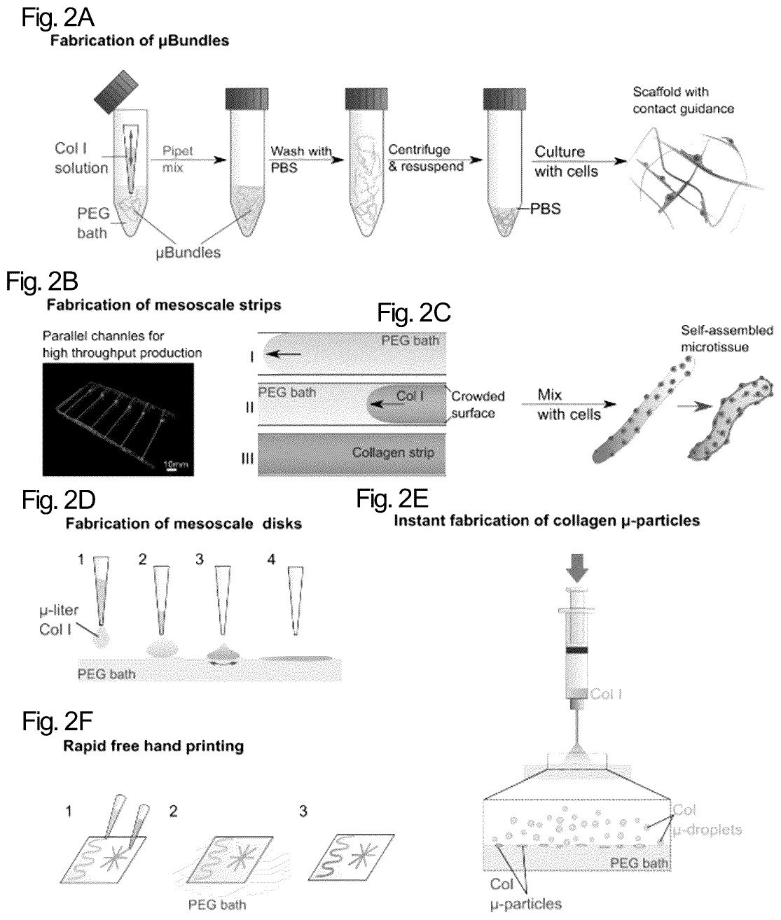

In step 104, the cellular solution is delivered into the macromolecular bath solution. In one embodiment, the cellular solution may be delivered by any method known to one skilled in the art to form various shapes (FIG. 2).

Aspects of the present invention relate to materials for the macromolecular bath solution. In some embodiments, the macromolecular bath solution comprises PEG of any concentration. In some embodiments, the macromolecular bath solution comprises PEG of concentrations from 0 to 1000 mg/mL. In some embodiments, the macromolecular bath solution comprises PEG of molecular weight from 0 to 2000 Da. In some embodiments, the macromolecular bath solution comprises PEG of any molecular weight. In some embodiments, the macromolecular bath solution comprises other molecular crowding agents. In some embodiments, the macromolecular bath solution is mixed with other materials or fluids, such as a granular support bath (e.g. agarose slurry, gelatin slurry) or a viscosity-modifying reagent such as methylcellulose.

In one embodiment, the macromolecular bath solution may be sterilized with any method known to one skilled in the art including but not limited to being sterilized by a 0.45 μm syringe filter.

In one embodiment, the macromolecular bath solution may be stored at any temperature known to one skilled in the art. In one embodiment, the macromolecular bath solution may be stored in room temperature.

Aspects of the present invention relate to materials for a collagen solution. In some embodiments, the collagen solution is mixed with other materials, such as other extracellular matrix proteins or proteoglycans, including fibrinogen, thrombin, fibrin, collagen IV, laminin, basement membrane proteins, Matrigel, Geltrex, and any combinations thereof at any concentrations. In some embodiments, the collagen solution is replaced by a solution of another extracellular matrix protein or proteoglycan, such as fibrin, fibrinogen, thrombin, collagen IV, laminin, basement membrane proteins, Matrigel, Geltrex, and any combinations thereof at any concentrations. In some embodiments, the collagen solution has any amount of collagen of any concentration. In some embodiments, the collagen solution has low collagen concentration, such as 0 to 2 mg/mL. In some embodiments, the collagen solution has moderate collagen concentration, such as 2 to 6 mg/mL. In some embodiments, the collagen solution has high collagen concentration, such as 6 to 30 mg/mL. In some embodiments, the collagen solution has very high collagen concentration, such as above 30 mg/mL.

In one embodiment, any known collagen may be used in the methods described herein. A collagen may be any collagen known in the art, such as one of collagen Type 1-29. In one embodiment, the collagen may be a fibrillar collagen such as Types I, II, III, V and XI, which serve as a principal structural component in load-bearing extracellular matrix (ECM). The collagen may be isolated or derived from a natural source or manufactured in any suitable manner. For example, the collagen may be biochemically or synthetically manufactured, produced through genetic engineering, or the like. Collagen may also be purchased from any one of a number of commercial vendors. In one embodiment, Collagen I may be used to create scaffolds. Collagen may be obtained from any suitable mammalian tissue. For example, collagen may be obtained from tendons, bones, cartilage, skin, or any other suitable organ. In some embodiments, collagen is obtained from rat tail tendon, porcine or calf skin.

Regardless of the source, the collagen may be purified. The purified collagen may be in any suitable form, such as a powder.

The reconstituted collagen solution may be neutralized in any suitable manner. For example, the reconstituted collagen solution may be neutralized by adding a base to the solution. Any suitable base may be used. For example, the base may be sodium hydroxide. In one embodiment, the solution is neutralized by adjusting the pH of the solution to a pH of 5 or greater. In one embodiment, the solution may be adjusted to a pH of about 5 to about 10, such as about 5.5 to about 9.5, about 6 to about 9, about 6.5 to about 8.5, or about 6.5 to about 8. In one embodiment, the solution is neutralized by adjusting the pH of the solution to a pH of 7.

The neutralized solution may also be altered in any other suitable manner. For example, a suitable buffer, such as phosphate buffered saline or the like, may be added to the solution.

In one embodiment, the neutralized solution may comprise any suitable concentration of collagen. In one embodiment, the neutralized solution comprises collagen at a concentration of about 50 mg/m or less, such as about 30 mg/ml or less, about 25 mg/ml or less, about 20 mg/ml or less, about 15 mg/ml or less, about 10 mg/ml or less, about 8 mg/ml or less, about 6 mg/ml or less, about 4 mg/ml or less, or about 2 mg/ml or less.

In some embodiments, the cellular solution (e.g. collagen solution) used in the fabrication can be non-neutralized (e.g. acid solubilized or pepsin solubilized). In some embodiments, the cellular solution (e.g. collagen solution) can be acidified in acetic acid or hydrochloric acid (HCl).

Aspects of the present invention relate to providing one or more cells to the collagen solution. In some embodiments, cells are mixed into the collagen solution before patterning with the crowding bath to generate cell-laden collagenous tissues in desired patterns. In some embodiments, the cells are induced pluripotent stem cells, which can maintain pluripotency. In some embodiments, the cells are induced pluripotent stem cells, which can be induced to differentiate into different cell types in situ, forming differentiated tissues in the user-designable patterns. In some embodiments, the patterned stem cells are differentiated into intestinal lineage. In some embodiments, the cells are stem cells of any type or form. In some embodiments, the cells are differentiated from stem cells. In some embodiments, the cells are adult or mature cell types. In some embodiments, cell clusters or organoids are mixed into the collagen solution before patterning with the crowding bath to generate cell-laden collagenous tissues in desired patterns. In some embodiments, the cells are spatially patterned. In some embodiments, different cell types are spatially patterned. In some embodiments, the cells are cancer cells. In some embodiments, the cancer cells are mixed with the collagen fiber bundles to form tumor spheroids or organoids with fibrotic features. In some embodiments, the cells are mixed with the collagen fiber bundles to form cell-matrix structures, with potential for guided morphogenesis.

As explained herein, the skilled person understands that the methods of the current invention may be applicable to different plant cells, for example plant cells of different plant species. It is contemplated the inventions disclosed herein may be applicable to plant cells of a wide range of plants, both monocots and dicots. Non-limiting examples include plant cells from the Cucurbitaceae, Solanaceae and Gramineae, maize/corn (Zea species), wheat (Triticum species), barley (e.g. Hordeum vulgare), oat (e.g. Avena sativa), sorghum (Sorghum bicolor), rye (Secale cereale), soybean (Glycine spp, e.g. G. max), cotton (Gossypium species, e.g. G. hirsutum, G. barbadense), Brassica spp. (e.g. B. napus, B. juncea, B. oleracea, B. rapa, etc), sunflower (Helianthus annus), safflower, yam, cassava, alfalfa (Medicago sativa), rice (Oryza species, e.g. O. sativa indica cultivar-group or japonica cultivar-group), forage grasses, pearl millet (Pennisetum spp. e.g. P. glaucum), tree species (Pinus, poplar, fir, plantain, etc), tea, coffea, oil palm, coconut, vegetable species, such as pea, zucchini, beans (e.g. Phaseolus species), cucumber, artichoke, asparagus, broccoli, garlic, leek, lettuce, onion, radish, lettuce, turnip, Brussels sprouts, carrot, cauliflower, chicory, celery, spinach, endive, fennel, beet, fleshy fruit bearing plants (grapes, peaches, plums, strawberry, mango, apple, plum, cherry, apricot, banana, blackberry, blueberry, citrus, kiwi, figs, lemon, lime, nectarines, raspberry, watermelon, orange, grapefruit, etc.), ornamental species (e.g. Rose, Petunia, Chrysanthemum, Lily, Gerbera species), herbs (mint, parsley, basil, thyme, etc.), woody trees (e.g. species of Populus, Salix, Quercus, Eucalyptus), fibre species e.g. flax (Linum usitatissimum) and hemp (Cannabis sativa), or model organisms, such as Arabidopsis thaliana. Further, the population of plant cells is a population of plant protoplasts of any plant type including those mentioned herein.

Aspects of the present invention relate to providing one or more cells to the cellular solution. In some embodiments, the one or more cells comprise any of, but not limited to, parenchymal cells, collenchyma cells, sclerenchyma cells, xylem cells, phloem cells, meristematic cells, epidermal cells, chloroplasts, plastids, leucoplasts, chromoplasts, plasmodesma, amyloplast, guard cells, vessel element cells, and sieve tube element cells.

Aspects of the present invention relate to forming tissue in various patterns and shapes. In some embodiments, the present invention provides a method for forming cellular tissue (e.g. collagenous tissue). In some embodiments, the formed cellular tissue (e.g. collagenous tissue) is a macroscopic pattern (millimeters and above in scale). In some embodiments, the formed cellular tissue (e.g. collagenous tissue) is comprised of multiple fibers (e.g. fiber-like bundles of collagen). with diameters ranging from 0 to 1000 micrometers, which can be accomplished by applying mechanical mixing, such as with a pipette, of the cellular solution (e.g. collagen solution) with the crowding solution or macromolecular bath. In some embodiments, the formed cellular tissues (e.g. collagenous tissues) are disk shaped. In some embodiments, the patterned cellular tissues (e.g. collagenous tissues) are in the form of an elongated or strip geometry. In some embodiments, collagen of different architectures (such as collagen fiber bundles), are mixed together to form a heterogenous or hybrid material. In some embodiments, patterned tissues strips can be generated by filling or coating fluidic channels with the macromolecular bath solution and then flushing the cellular solution (e.g. collagen solution) through the same channels. In some embodiments, the cellular solution (e.g. collagen solution) is gelled or solidified rapidly (within seconds) upon contact or mixing with the macromolecular bath. In some embodiments, the cellular solution (e.g. collagen solution) is neutralized with a pH neutralizing or buffering solution. In some embodiments, the cells and/or collagen is solubilized, such as by acid or pepsin.

Aspects of the present invention relate to tools, equipment and/or devices for forming cellular tissue (e.g. collagenous tissue). In some embodiments, fluid channels are used for molding the patterned tissue. In some embodiments, a pipette or syringe or other fluid extruder is used to deposit cellular solution (e.g. collagen solution) into a macromolecular bath or crowding solution to create patterns. In some embodiments, an aerosol spraying contraption or device is used to spray cellular solution (e.g collagen solution) droplets or vapor into the crowding solution to create micrometer-scale droplet or emulsion patterns. In some embodiments, a translatable or movable fluid extruder is used to extrude the cell solution (e.g. collagen solution) in user-defined or designable patterns to generate cellular tissues (e.g. collagenous tissues) into spatial patterns.

Aspects of the present invention relate to tunable properties for the cellular solution (e.g. collagen solution). In some embodiments, the gelation speed of the cellular solution (e.g. collagen solution) is tunable, such as by tuning the cell and/or collagen concentration or the PEG concentration. In some embodiments, the gelation speed of the cellular solution (e.g collagen solution) is rapid, in seconds or minutes time scales, such as by using high concentration cell solution (e.g. collagen solution) or high concentration PEG.

In one embodiment, the cellular solution (e.g. collagen solution) may be delivered through a pipet tip to create cellular and/or collagen μ-bundles. In one embodiment, the pipet tip may be any suitable size known to one skilled in the art including but not limited to a 1000 μL pipet tip. In one embodiment, cellular and/or collagen I-bundle suspension can be incorporated into another hydrogel or mixed with cells to create a 3D tissue model with complex ECM architectures.

In one embodiment, the cellular solution (e.g. collagen solution) may be delivered into microchannels to create cellular strips (e.g. collagen strips) (FIG. 2). In one embodiment, the microchannels may be molded in silicone polymer polydimethylsiloxane (PDMS) with needles. In one embodiment, the needles may be any suitable size. In one embodiment, the needles may be 500 μm in diameter. In one embodiment, the microchannels may be sterilized with any suitable method known to one skilled in the art including but not limited to being sterilized with 70% ethanol and rinsed with sterile PBS before use. In one embodiment, cells at varied densities can be mixed in the cellular solution (e.g. collagen solution) and fabricated into free-floating microtissue.

In one embodiment, the cellular solution (e.g. collagen solution) may be delivered as a droplet to form a microdisk. In one embodiment, the droplet may be formed at a tip of a micropipette (FIG. 2). In one embodiment, the microdisks can be incorporated with cells and washed in PBS to build microtissues.

In one embodiment, the droplets may be sprayed into the macromolecular bath solution to form microparticles. In one embodiment, the droplets may be sprayed into the macromolecular bath solution by plunging a syringe filled with a small amount of cellular solution (e.g. collagen solution) (FIG. 2E). When falling onto the macromolecular bath solution, these droplets (typically a few tens of micrometers in diameter) instantly polymerize into microparticles. Due to the expansion of the droplets on the macromolecular bath, these particles often exhibit oblate or disk shapes.

In one embodiment, the cellular solution (e.g. collagen solution) may be hand printed on a surface. In one embodiment, the surface may one including but not limited to a glass coverslip or a PDMS block. In one embodiment, the surface may be treated with oxygen plasma to render a hydrophilic surface. In one embodiment, the cellular solution (e.g. collagen solution) may be hand drawn onto the surface with a fine needle or a 10-μL micropipette tip (FIG. 2F). These surfaces with designed cellular and/or collagen patterns then can be carefully transformed onto the macromolecular bath solution and rapidly plunge into the macromolecular bath. When submerged in the macromolecular bath solution, the cellular and/or collagen patterns instantly polymerize.

Aspects of the present invention relate to cellular tissue (e.g. collagenous tissue) formed by any disclosed method. In some embodiments, the formed cellular tissue (e.g. collagenous tissue) comprises multiple fiber-like bundles, with diameters larger than 1000 micrometers. In some embodiments, endothelial cells are mixed with bundles to form vascular networks or vascularized tissues. In some embodiments, the fiber bundles are injectable. In some embodiments, the fiber bundles are injected into tissues.

Cellular and/or collagen material produced by the methods described herein may be used to engineer any suitable tissue. For example, the cellular and/or collagen material may be used to engineer soft tissue structures, cartilaginous structure, connective tissue, vascular tissue, bone tissue, and the like. The cellular and/or collagen material may be used to engineer soft tissue of the trachea, epiglottis, vocal cords, and the like. The cellular and/or collagen material may be used to engineer articular cartilage, nasal cartilage, tarsal plates, tracheal rings, thyroid cartilage, arytenoid cartilage. The cellular and/or collagen material may be used to engineer vascular grafts and components thereof. The cellular and/or collagen material may be used to engineer sheets for topical applications or for repair of organs such as livers, kidneys, and pancreas. The cellular and/or collagen material may be used to engineer bone, dental structures, joints, cartilage, skeletal muscle, smooth muscle, cardiac muscle, tendons, menisci, ligaments, blood vessels, stents, heart valves, corneas, ear drums, nerve guides, tissue patches or sealants, a filler for missing tissues, skin, or the like. The cellular and/or collagen material may be used to engineer intestinal tissue, or the like.

Aspects of the present invention relate to uses and/or applications for cellular tissue (e.g. collagenous tissue) produced by any disclosed method. In some embodiments, the injected bundles in tissues are used for therapeutic applications. In some embodiments, the injected bundles in tissues are used for tissue engineering applications. In some embodiments, the injected bundles are used to recruit cells in vivo. In some embodiments, the injected bundles are used for immunological applications. In some embodiments, the injectable bundles are used to generate porous scaffolds in vitro or in vivo. In some embodiments, the bundles are used to form mesoporous scaffolds, with pore sizes ranging from 1 to 1000 micrometers. In some embodiments, the generated tissues are used for drug screening, discovery, or development applications. In some embodiments, the generated tissues are used for cancer drug screening, discovery, or development applications. In some embodiments, the generated tissues are used for cancer drug screening, discovery, or development applications. In some embodiments, the generated tissues are used for vascular tissue engineering applications. In some embodiments, the generated tissues are used for vascular disease screening, drug screening, drug discovery, or drug development applications. In some embodiments, the generated tissues are used for vascular tissue engineering applications.

In some embodiments, any tissue construct of the present invention may be further differentiated in situ, cultured, matured, and/or biochemically, biophysically, biologically stimulated (or modified) subsequently over time.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art may, using the preceding description and the following illustrative examples, make and utilize the compounds of the present invention and practice the claimed methods. The following working examples therefore specifically point out exemplary embodiments of the present invention and are not to be construed as limiting in any way the remainder of the disclosure.

### Example 1: Programmable Shaping of Collagen Scaffolds Through Rapid Macromolecular Crowding

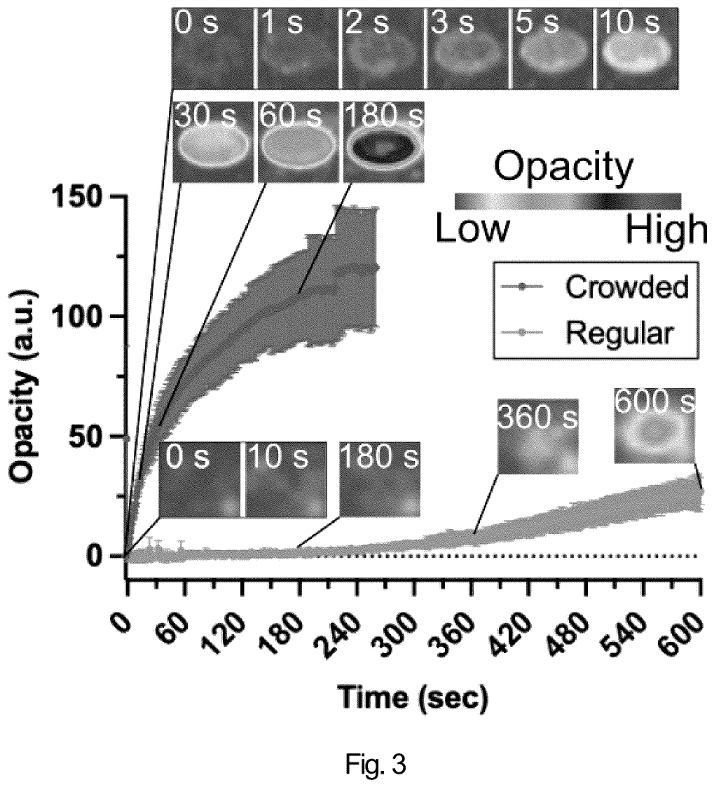

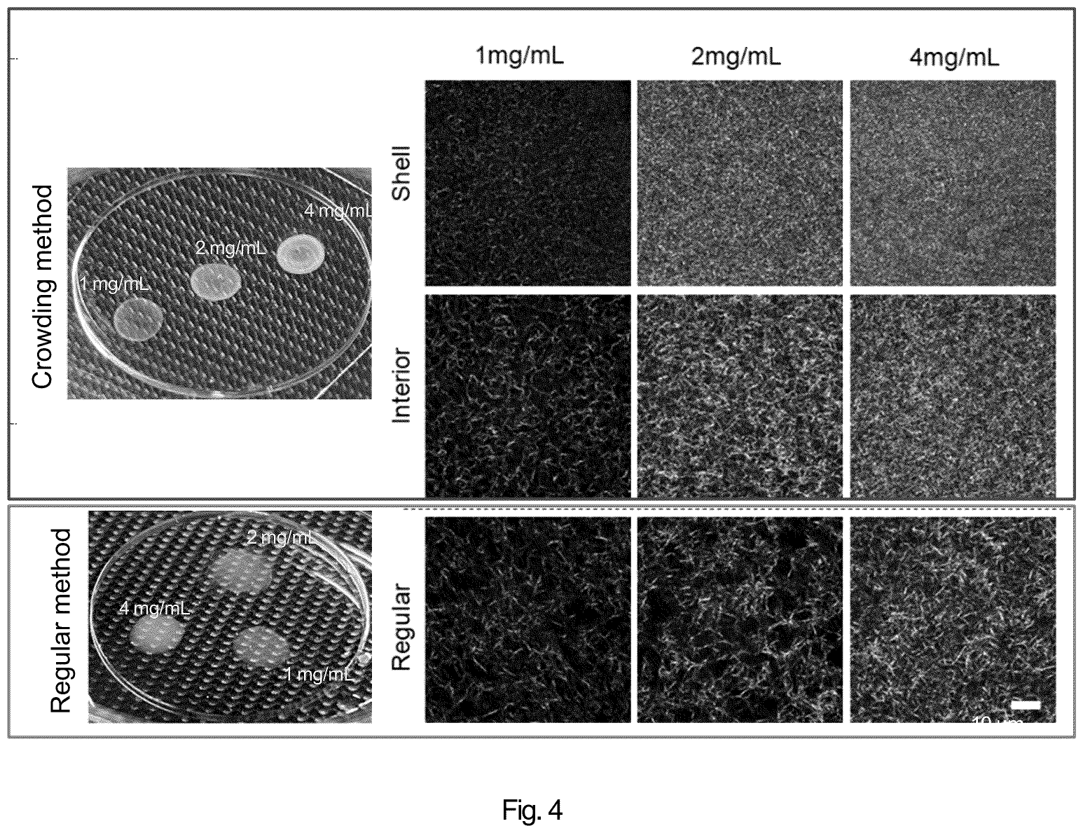

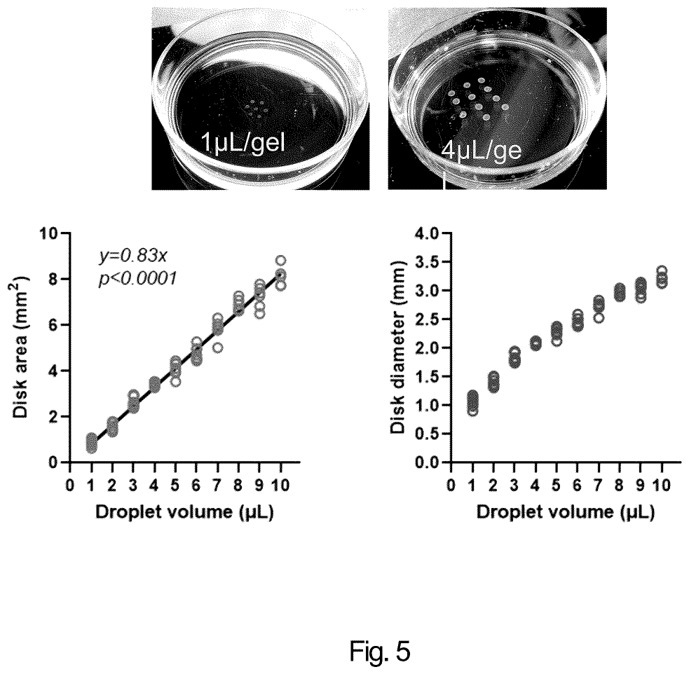

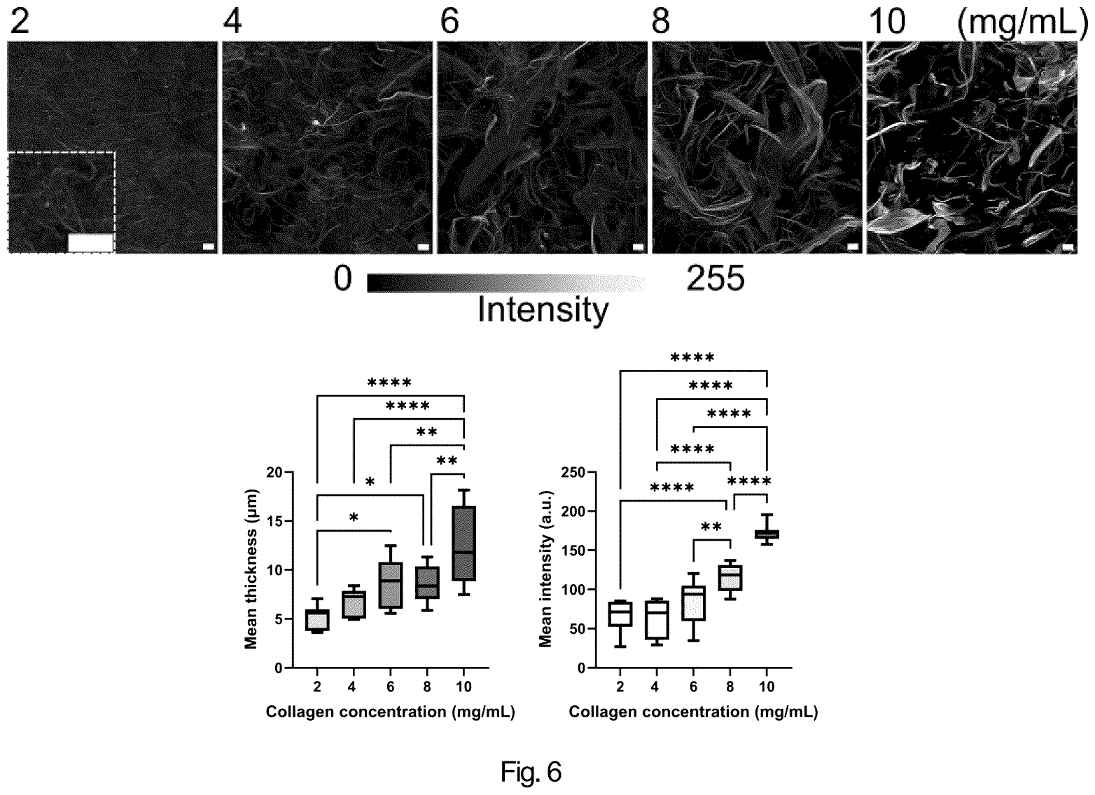

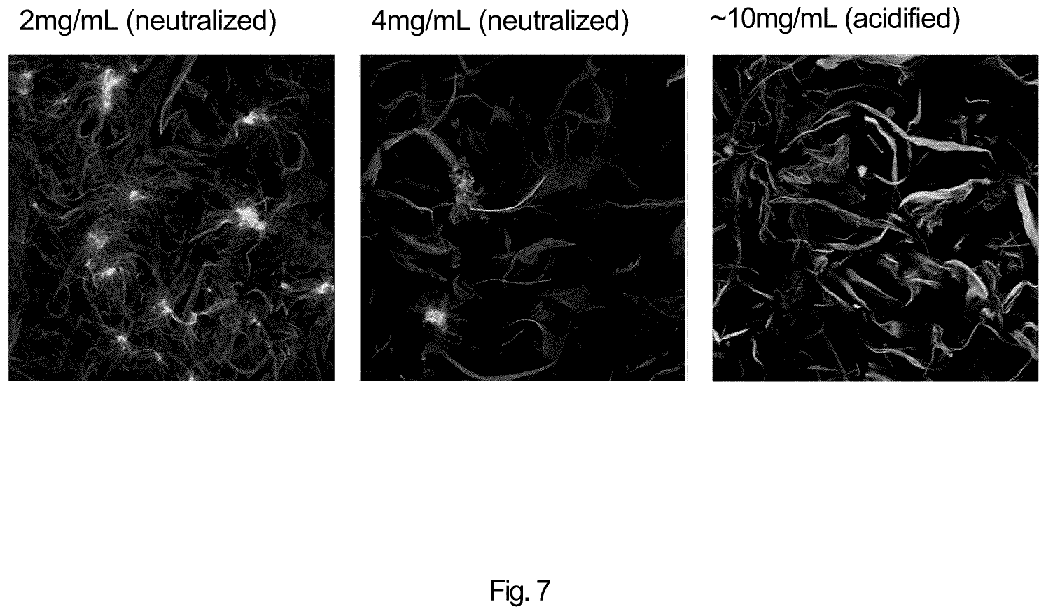

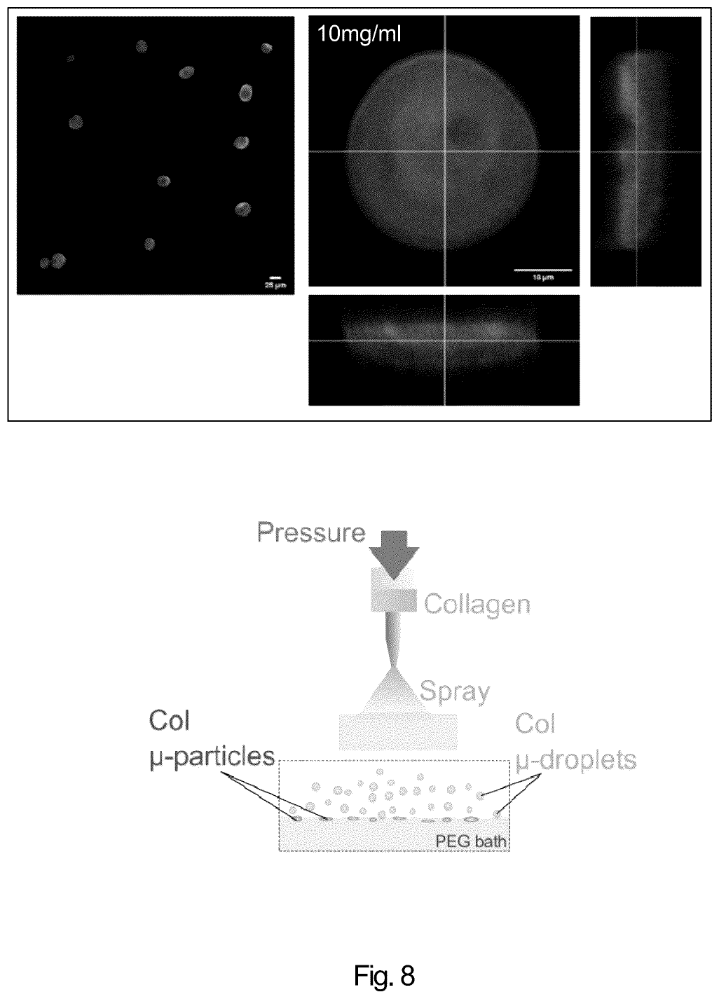

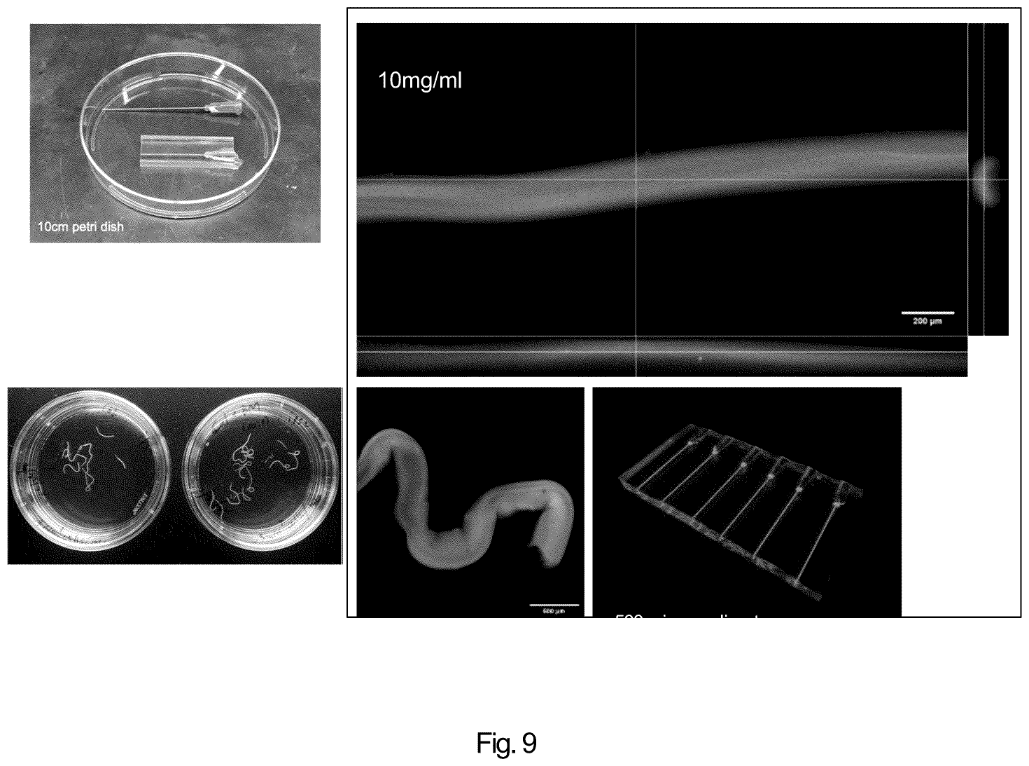

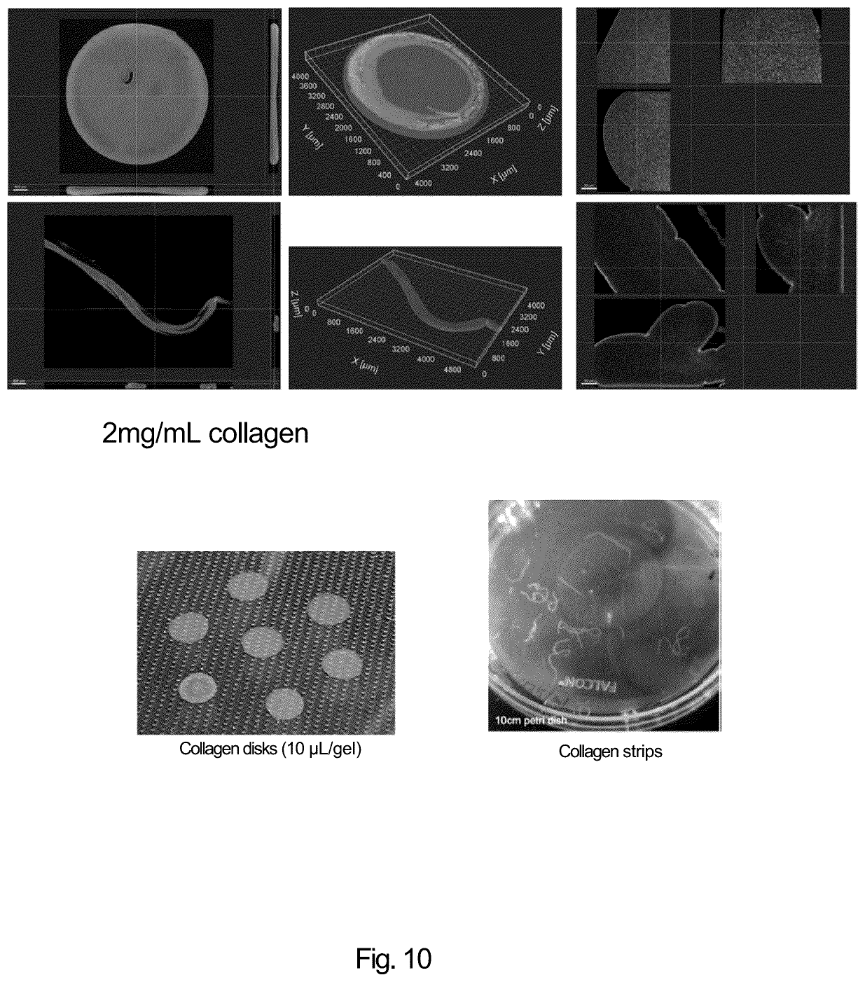

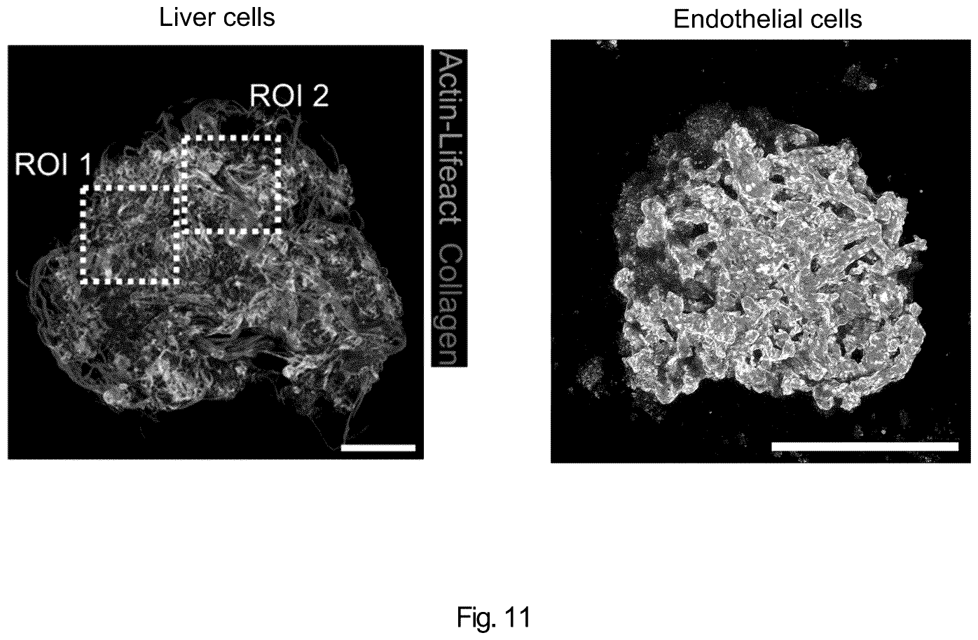

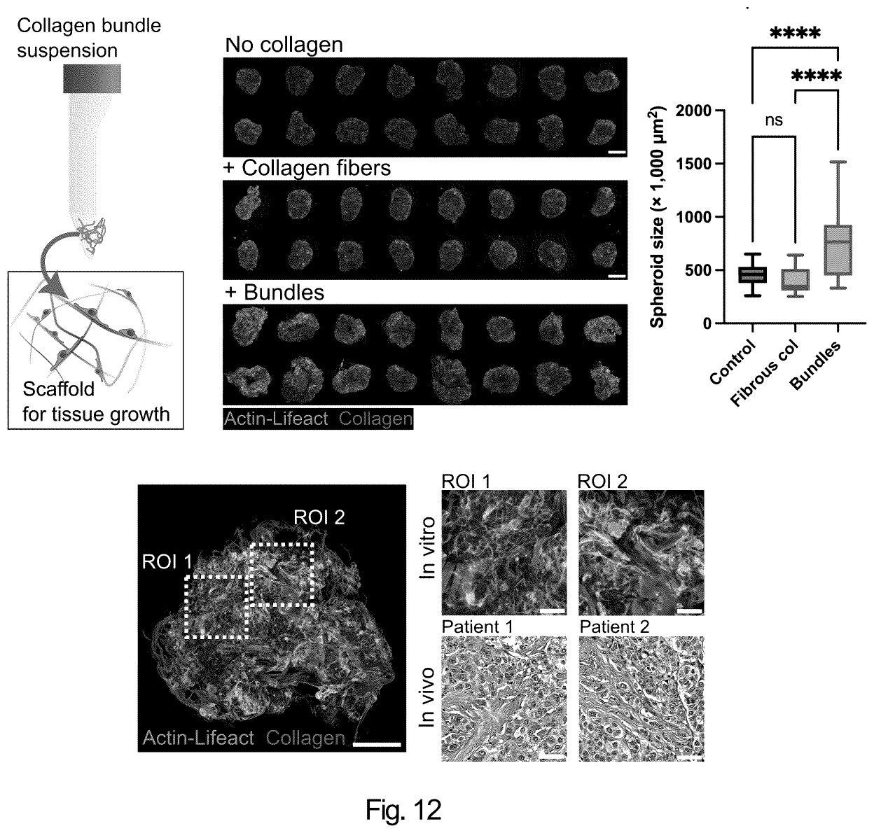

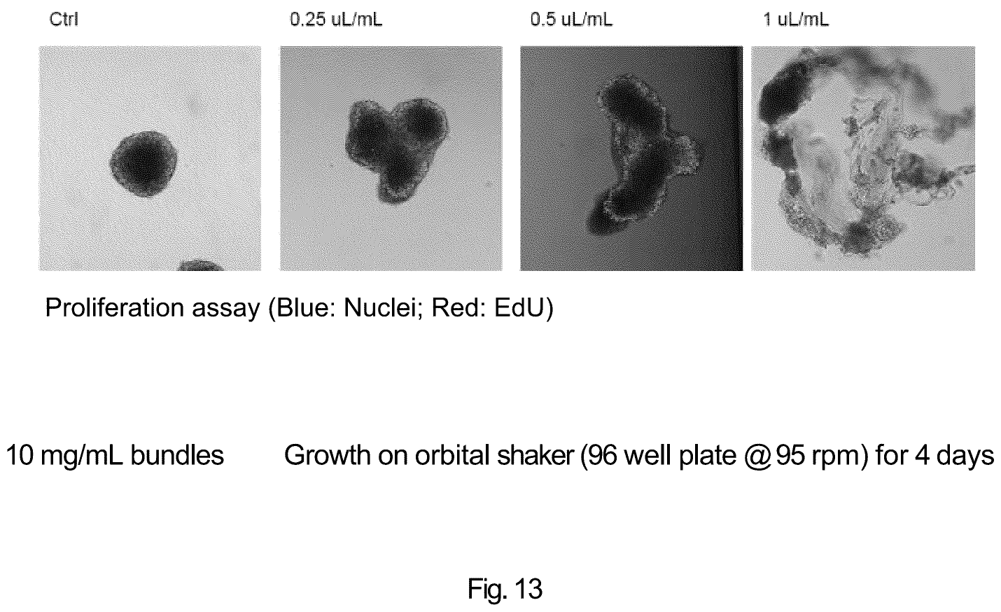

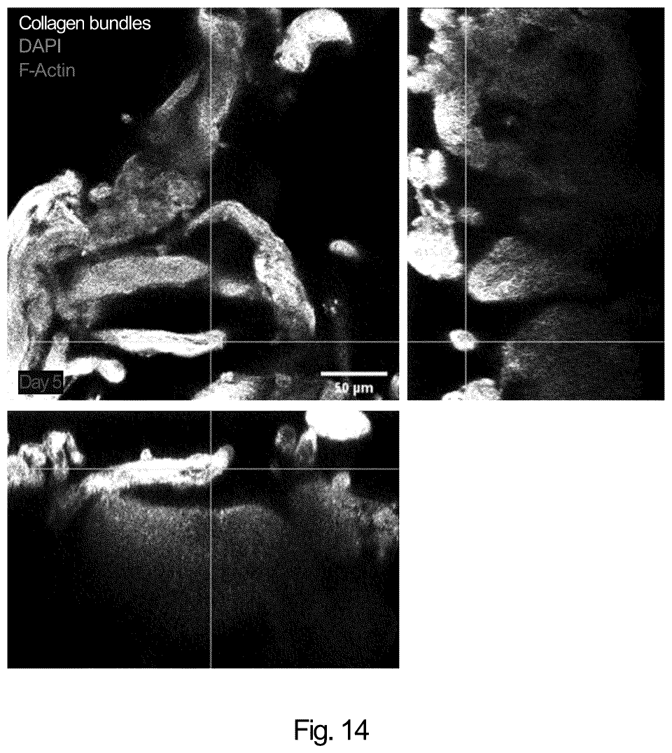

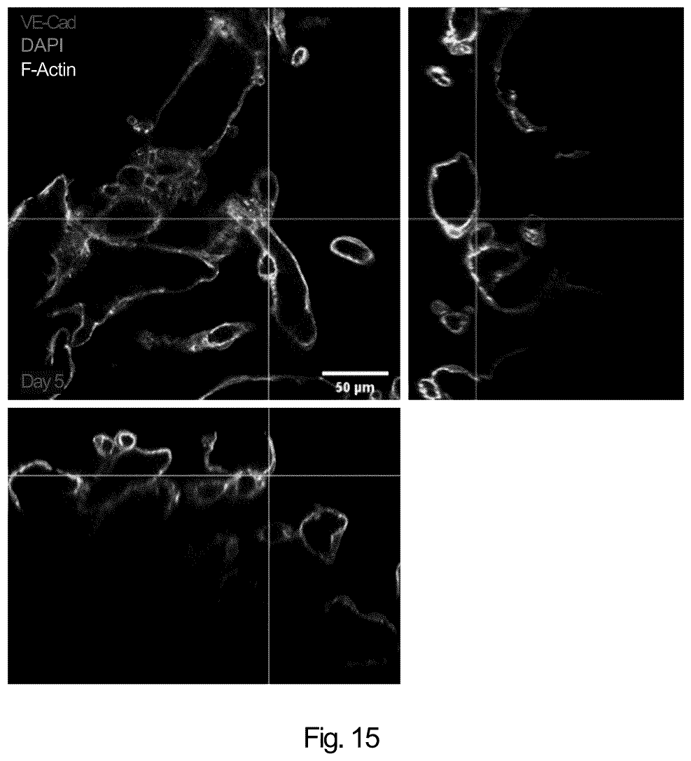

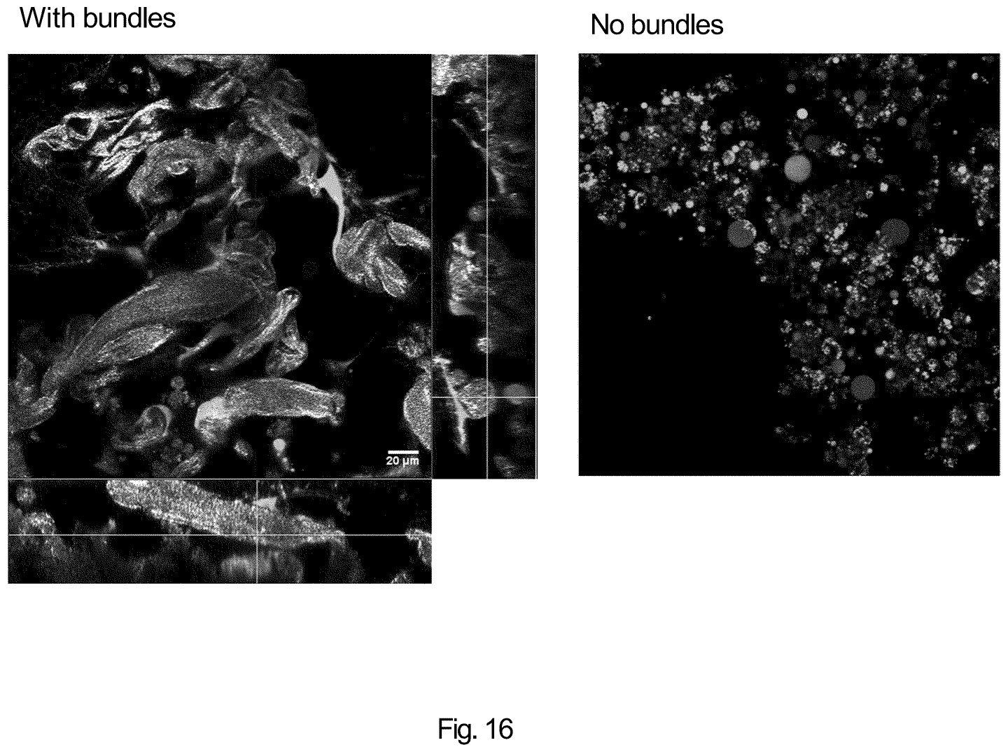

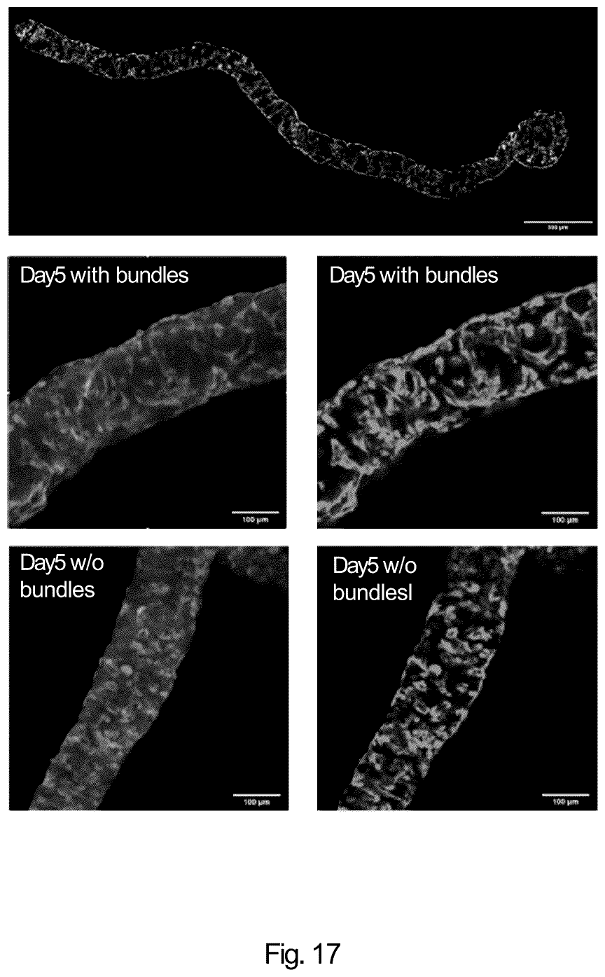

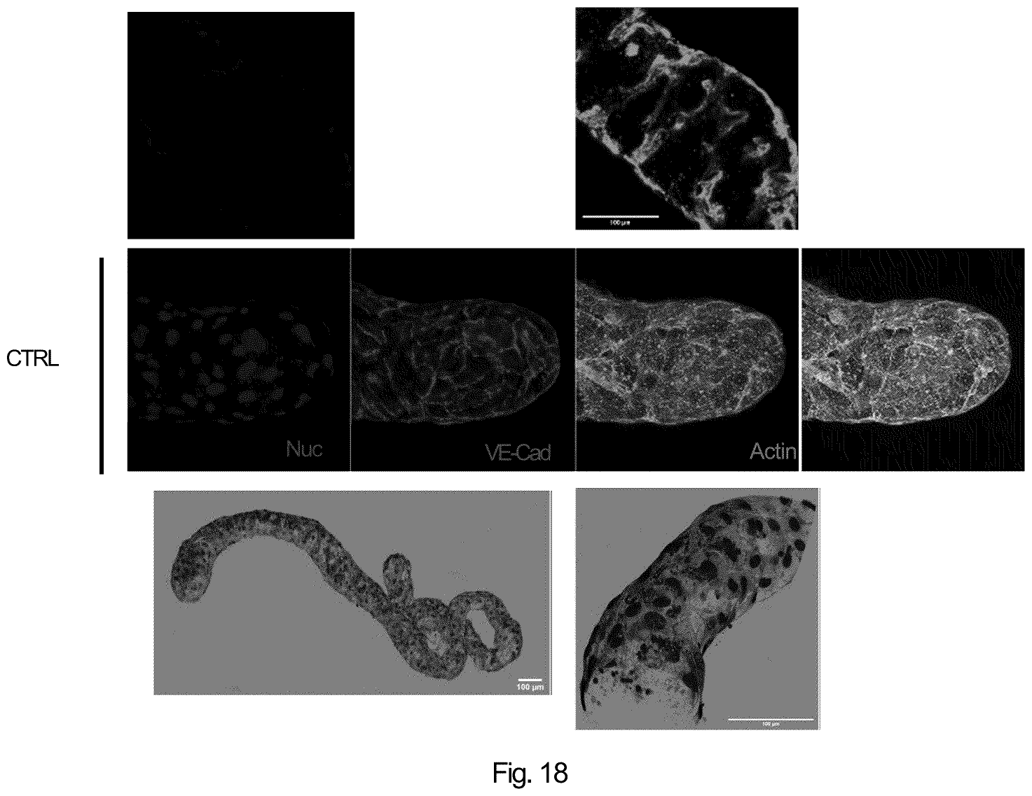

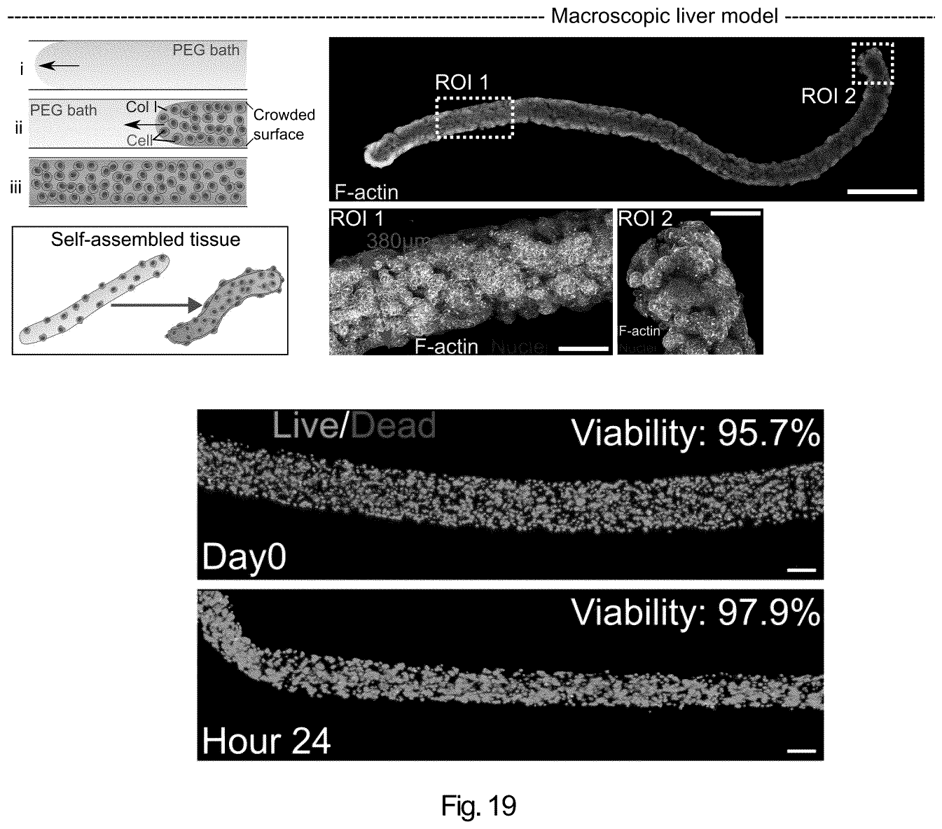

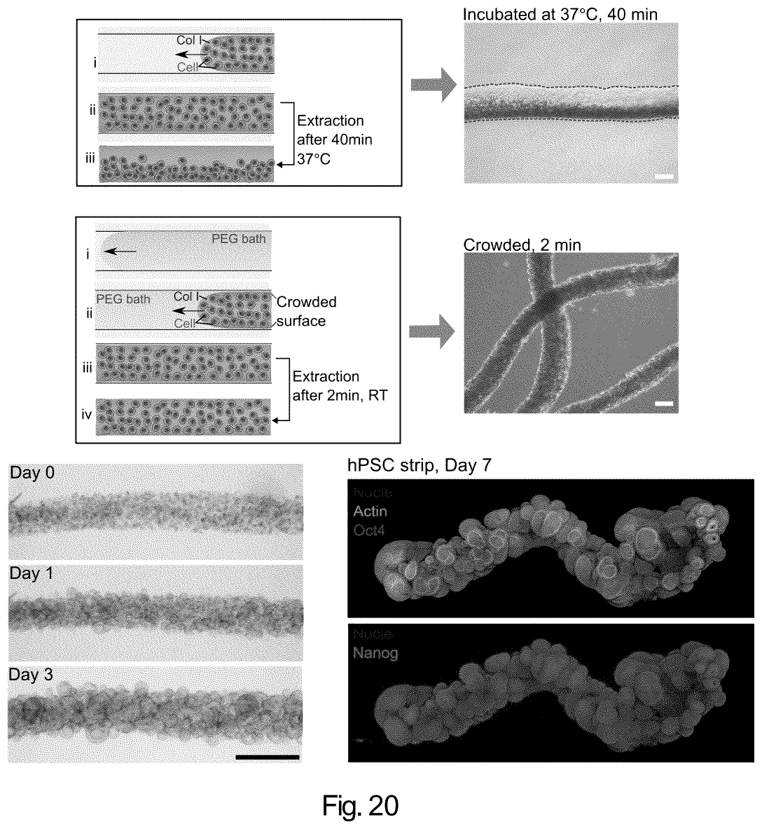

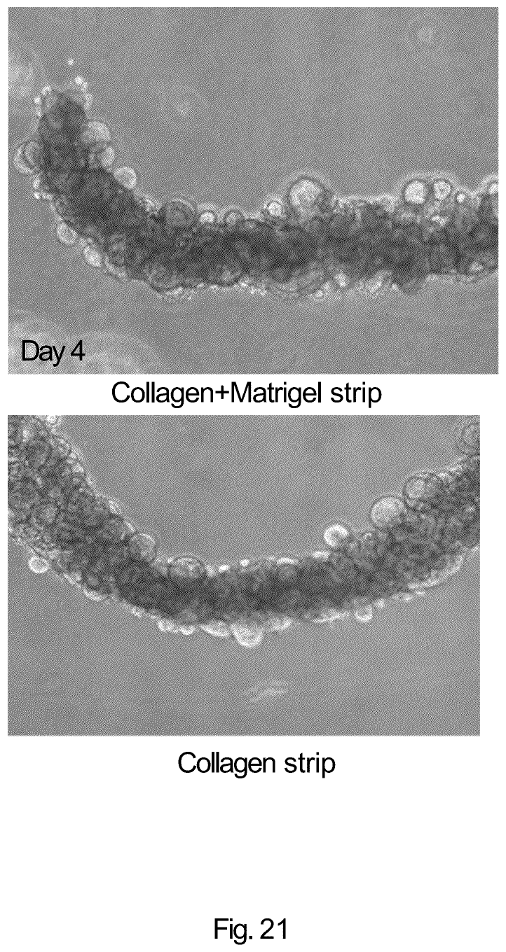

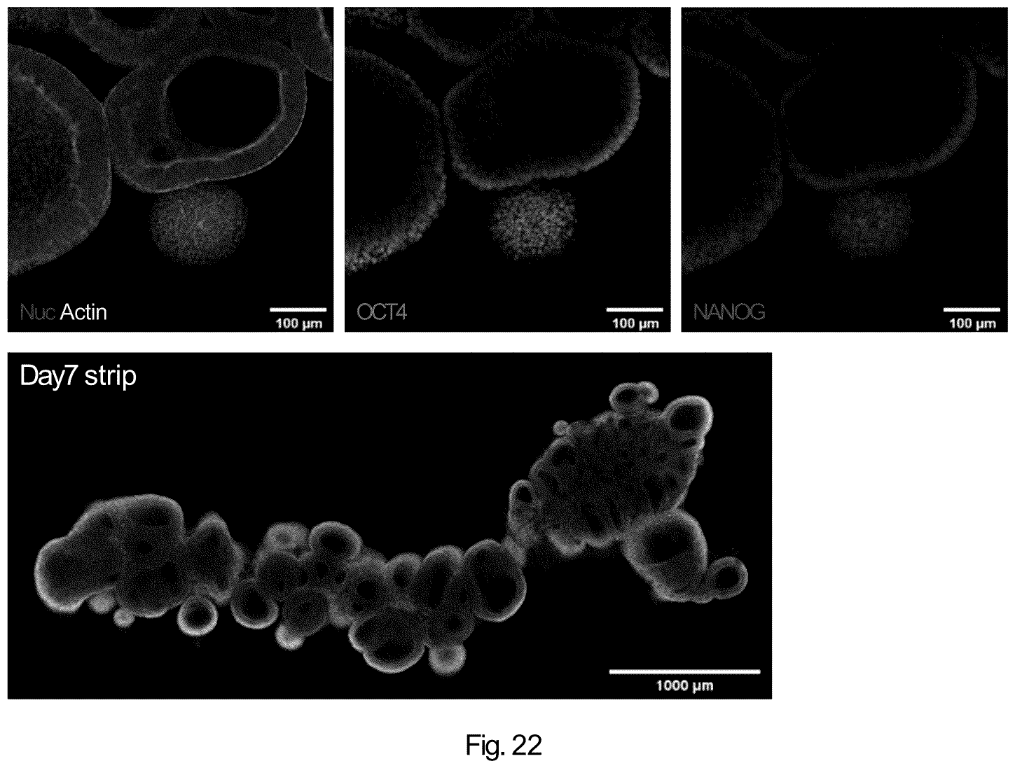

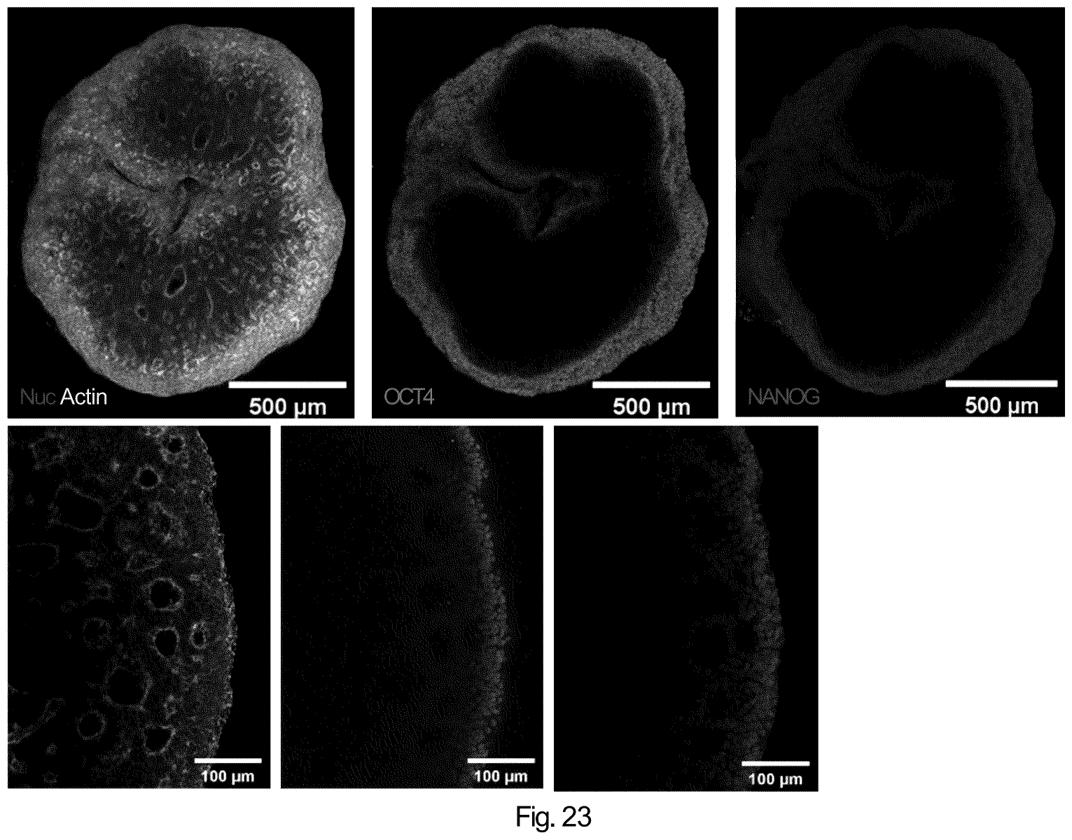

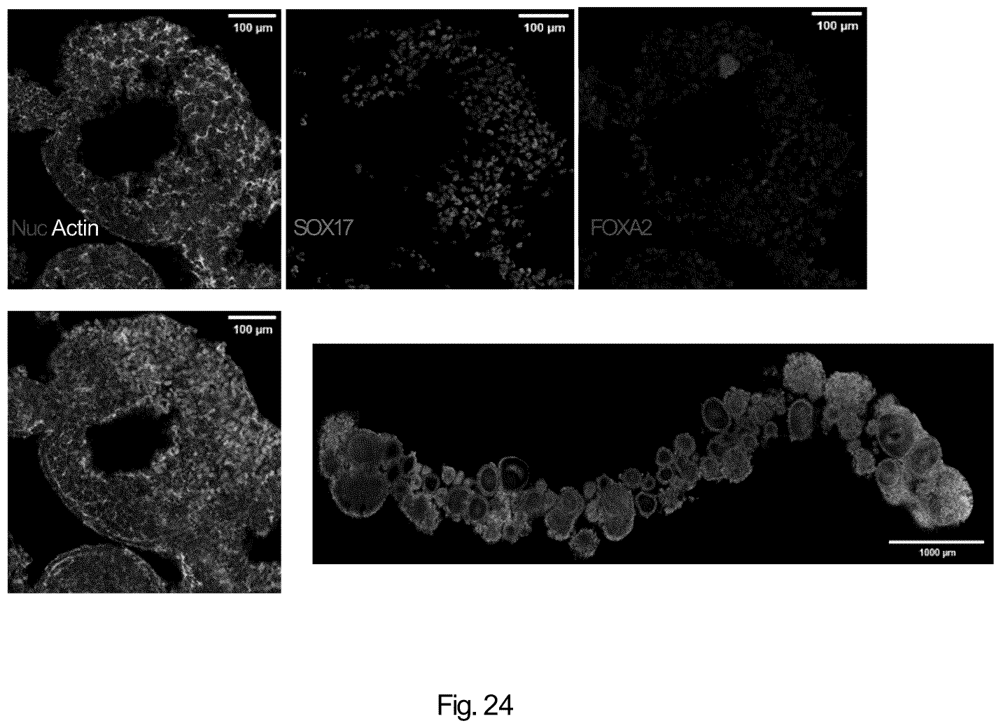

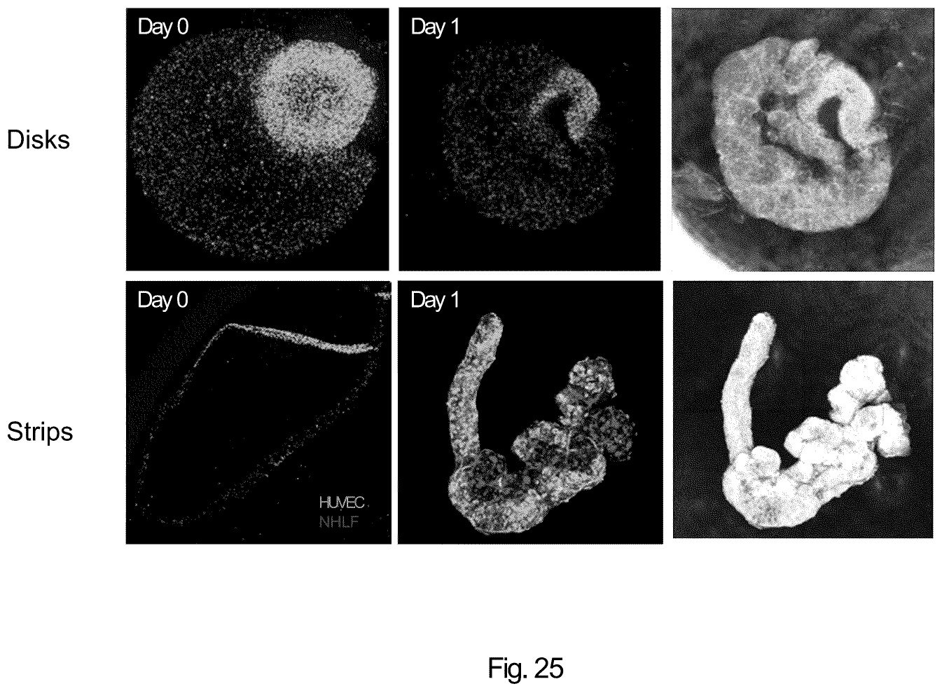

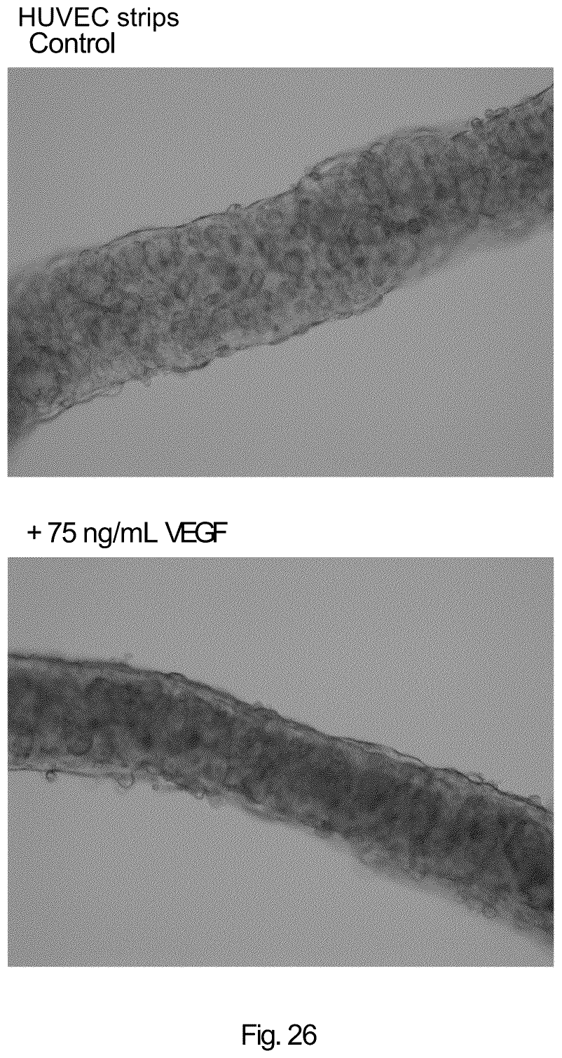

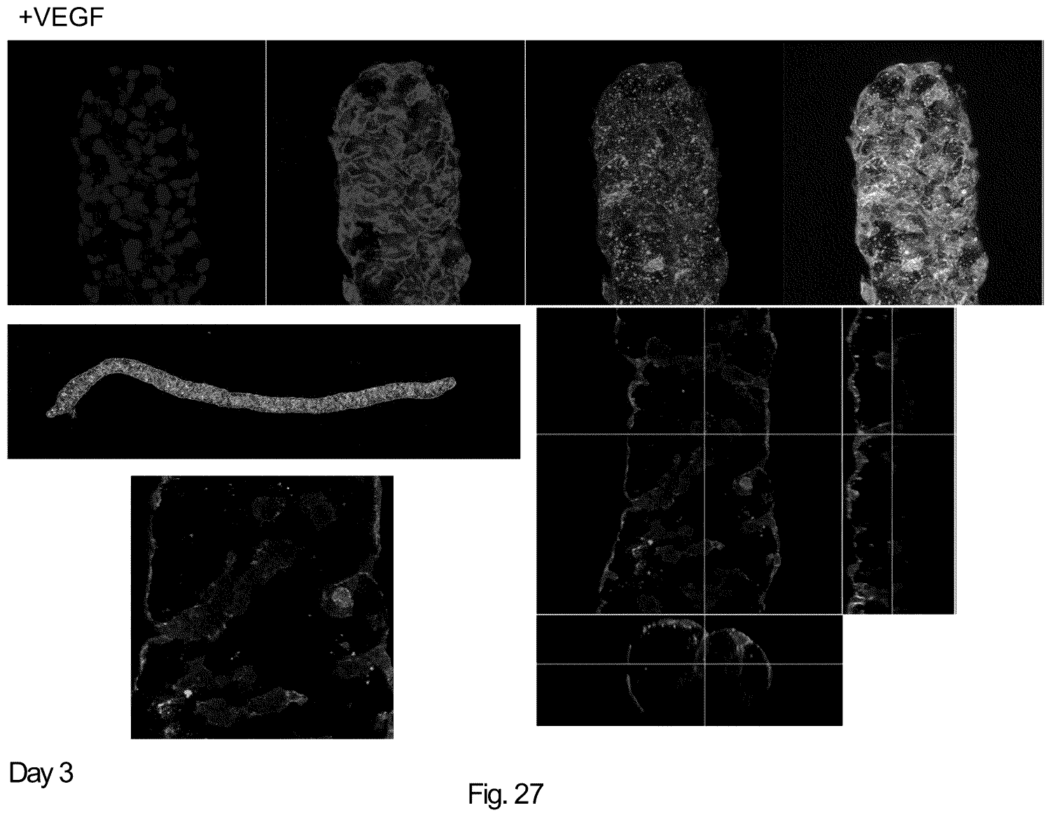

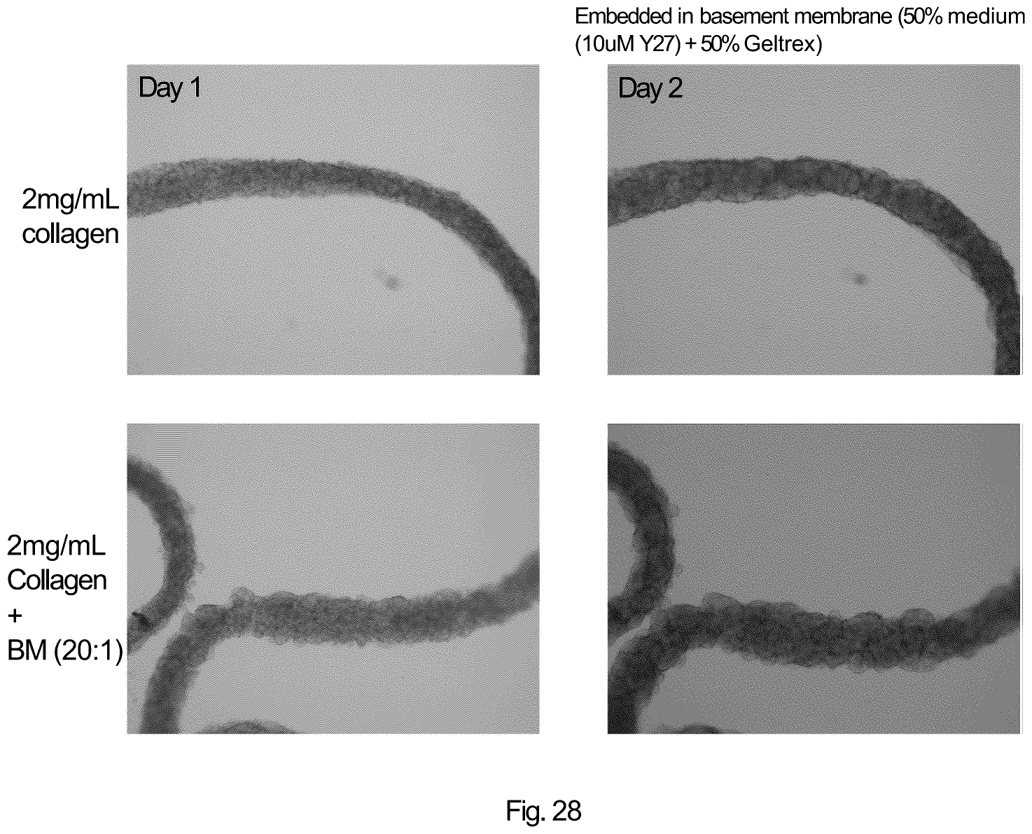

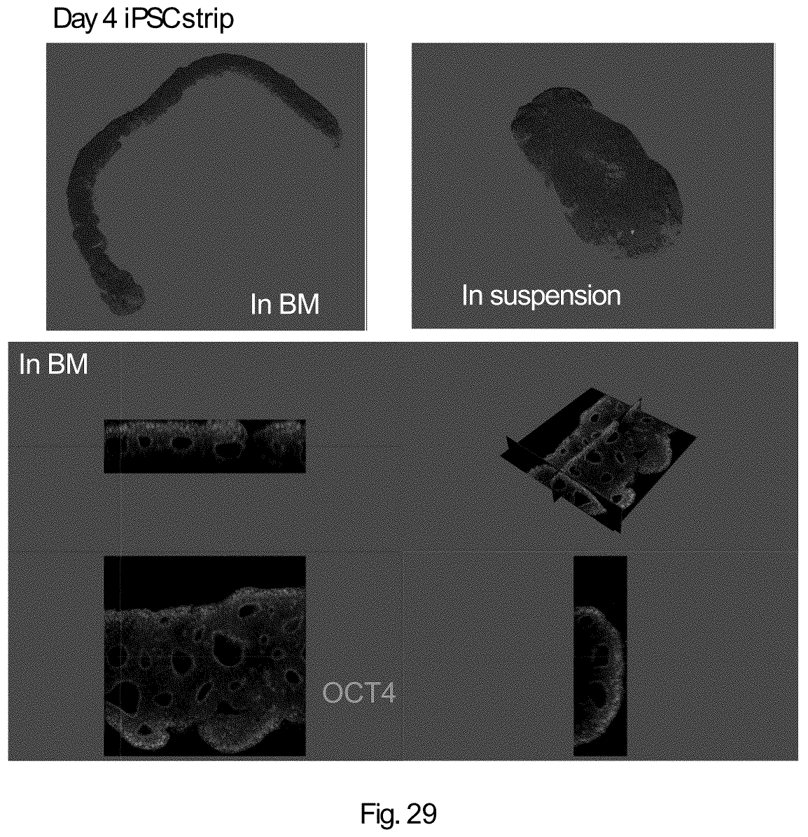

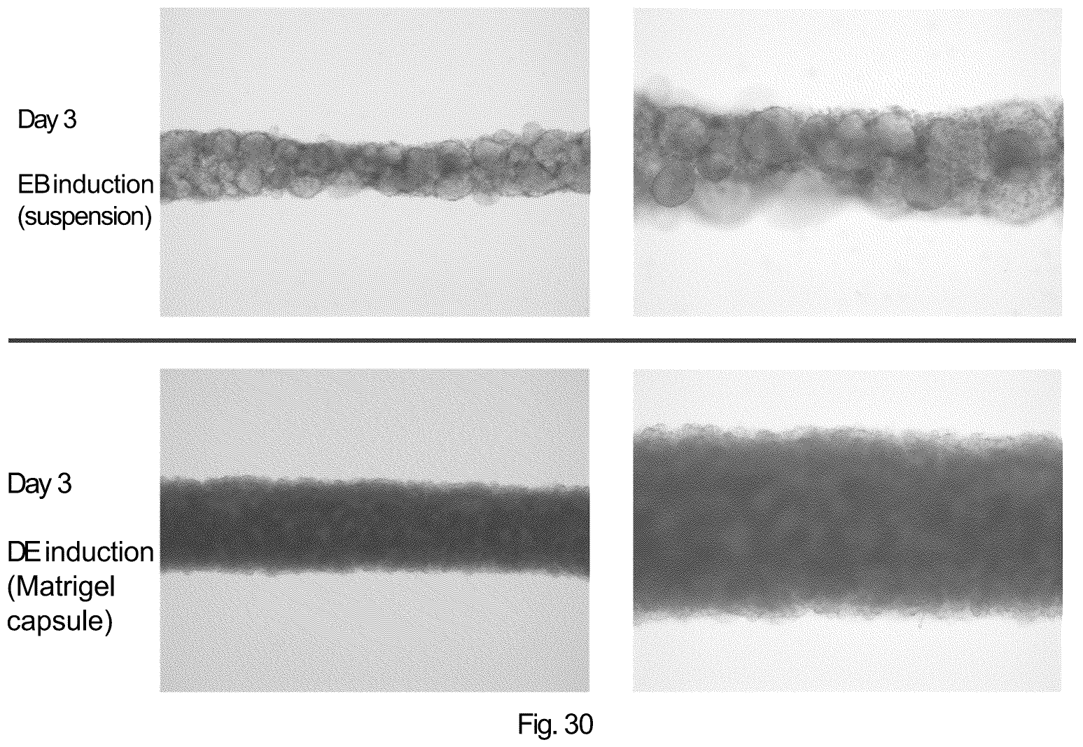

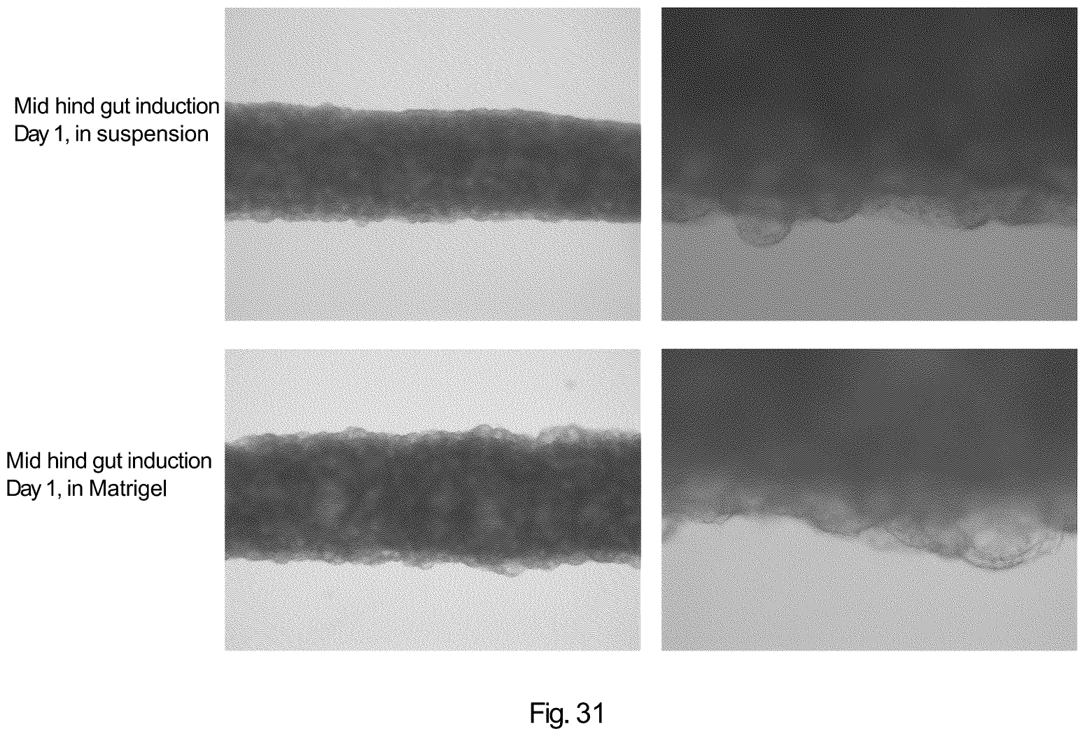

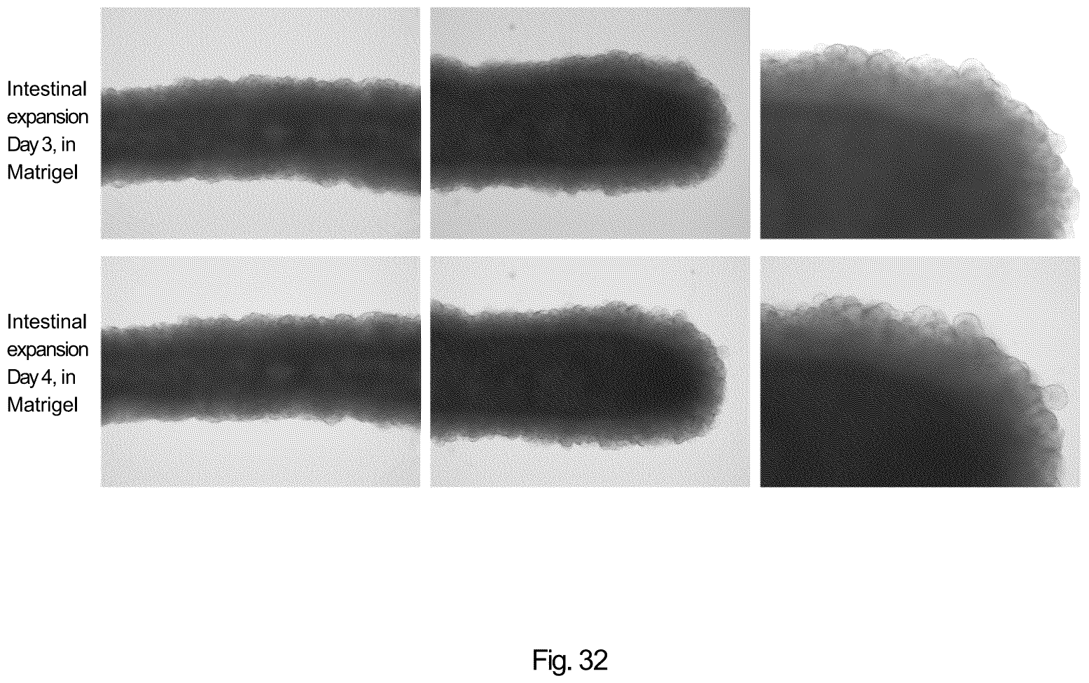

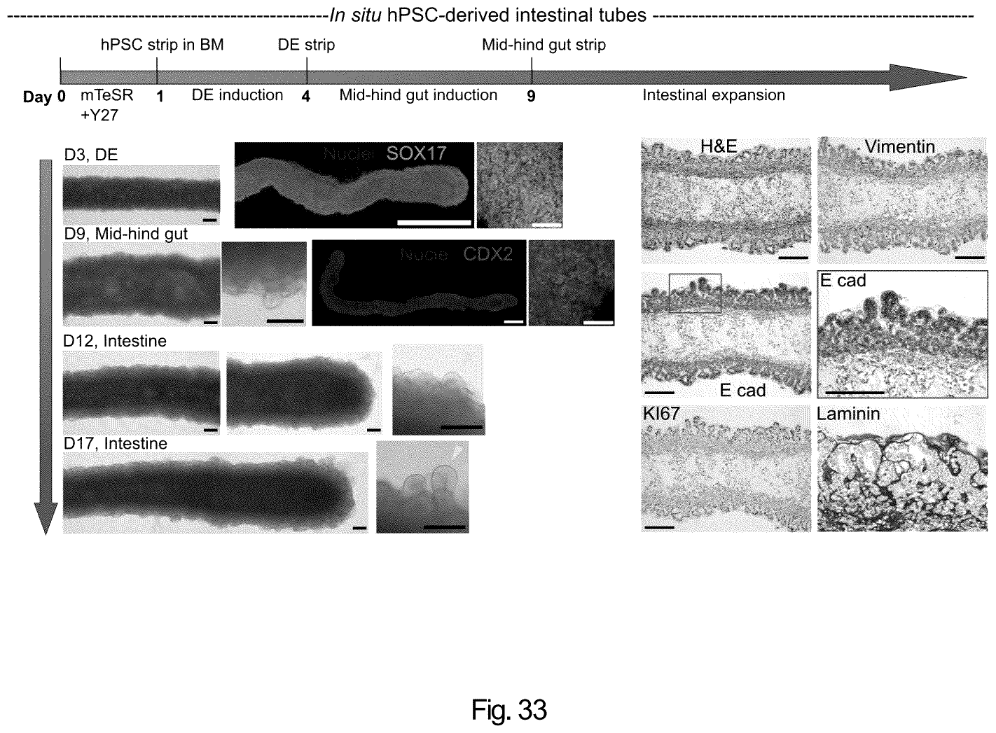

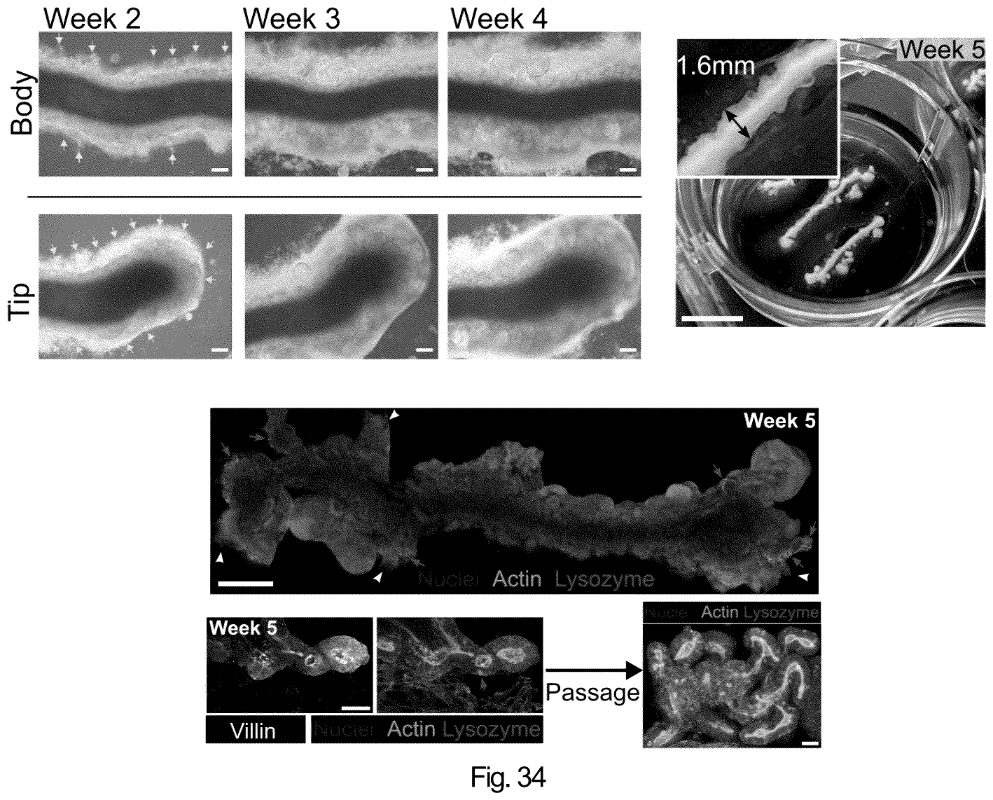

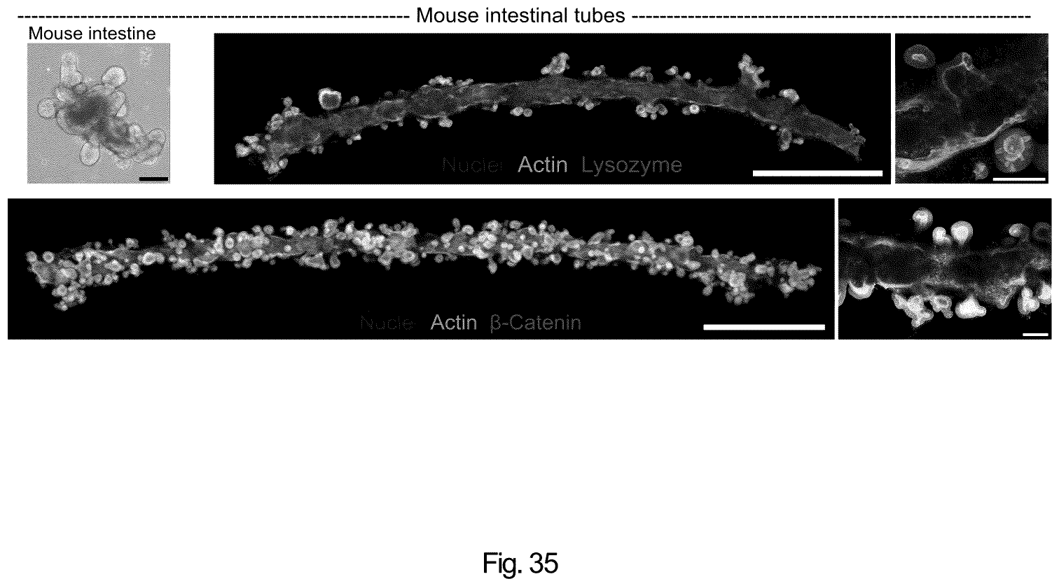

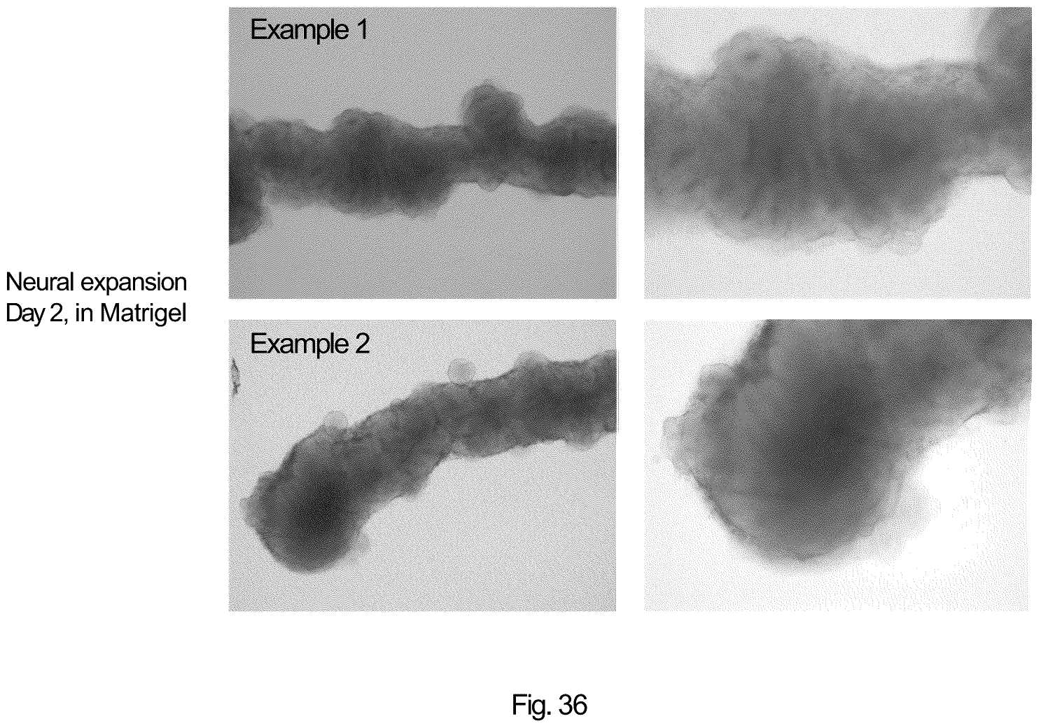

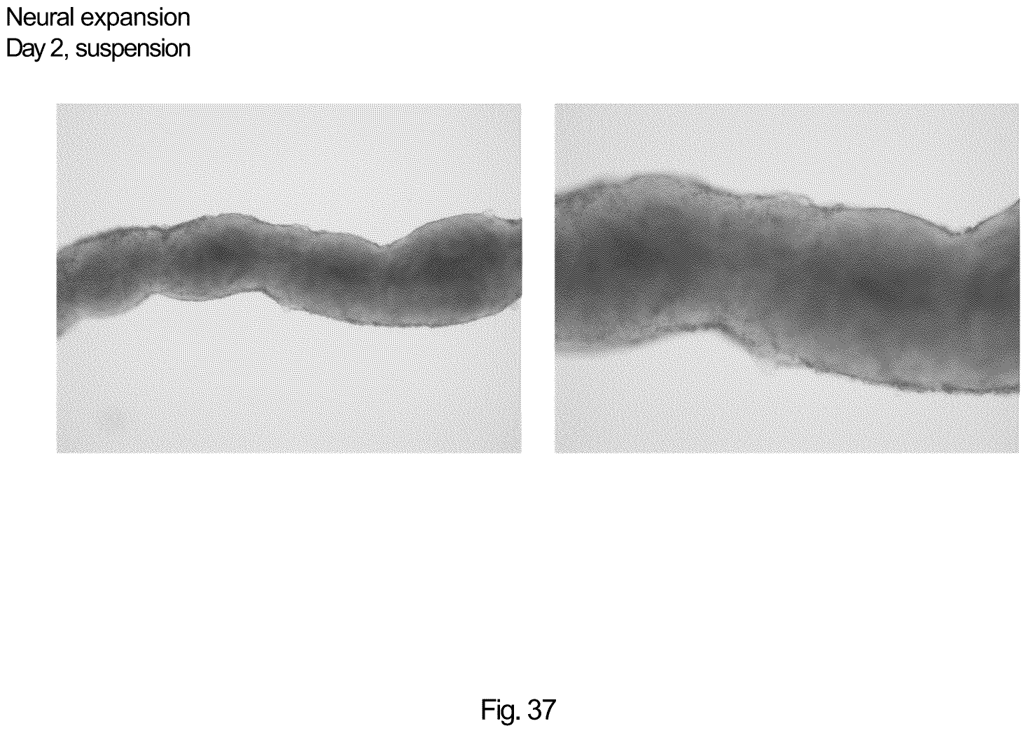

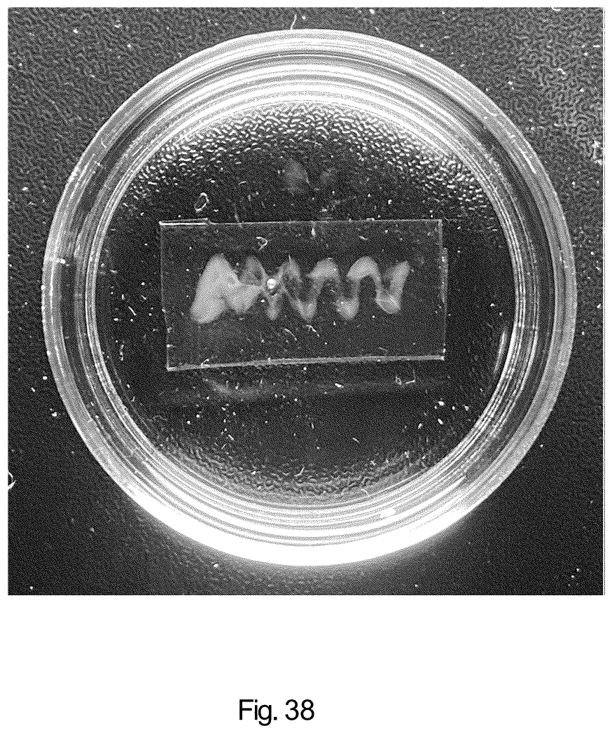

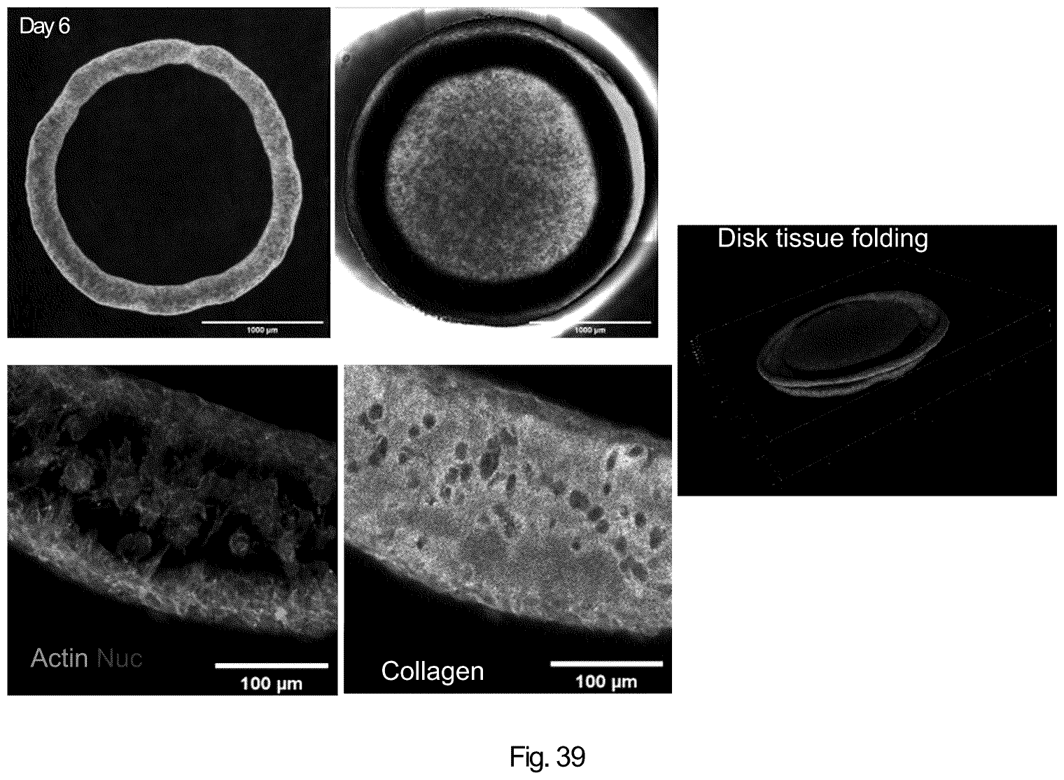

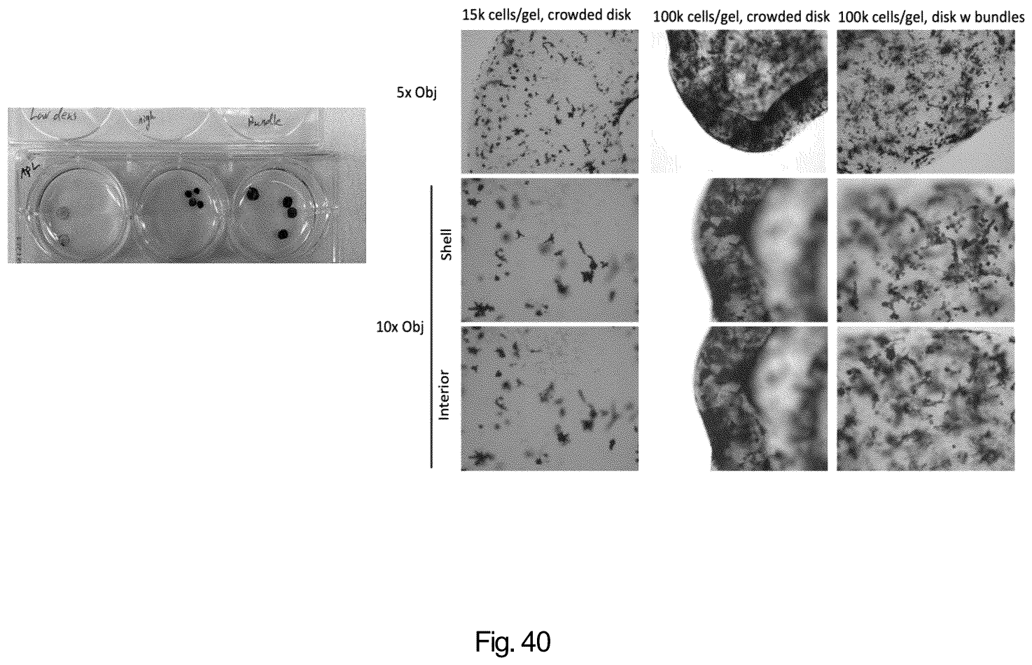

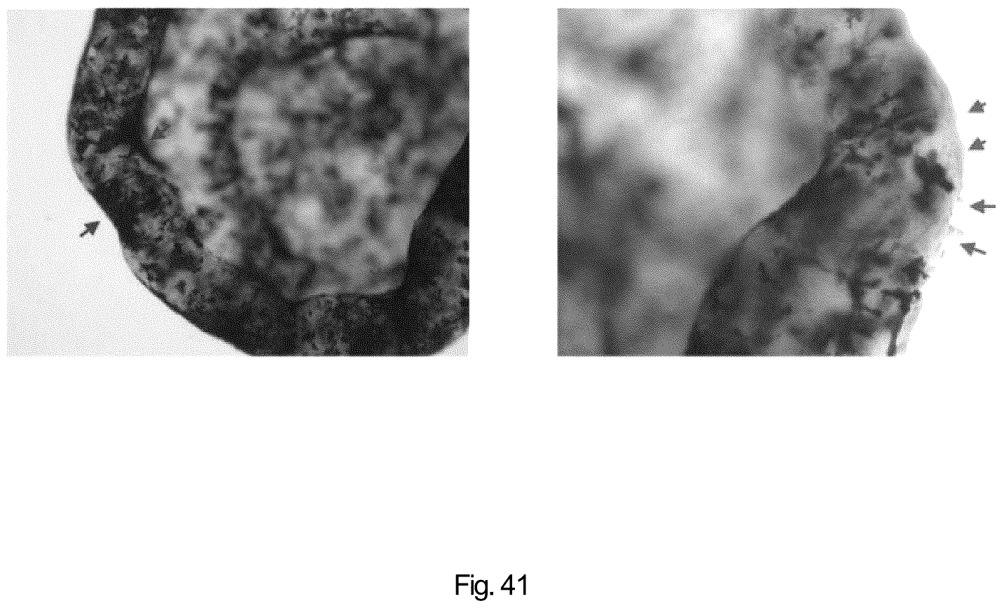

The materials and methods employed as well as the results of these experiments are now described and shown throughout FIG. 3 to FIG. 41.

### Rapid Gelation of Collagen I in Macromolecular Bath

The crowding bath was prepared by dissolving high molecular weight (˜8000 Da) polyethylene glycol PEG8000 (Sigma) in 1×PBS at the concentration of 200 mg/mL. The solution can be stored at room temperature. For tissue culture applications, the PEG8000 bath is sterilized by a 0.45 μm syringe filter. Rat tail collagen type I (Corning, 354249) is neutralized (pH=7) with NaOH and adjusted to the desired concentrations with PBS. When delivered into the PEG8000 bath, a collagen solution rapidly polymerizes and forms various shapes depending on the delivering methods (FIG. 2). Surprisingly, the crowding effect was seen to be able to override the low pH condition to polymerize the non-neutralized collagen rapidly. In this study, 2, 4, 6, 8 mg/mL and the non-neutralized (acidified) collagen (˜10 mg/mL) was used.

### Collagen μ-Bundle Fabrication in Macromolecular Bath

As shown in FIG. 2A, collagen solution (e.g., 100 μL) was rapidly pipetted into a PEG bath (3 mL) with a 1000 μL pipet tip and quickly mixed 20-25 times. Floating polymerized collagen bundles became visible in the PEG bath. To remove the PEG from the mixture before cell culture, the PEG bath was diluted, and the μ-bundles were washed with 12 mL PBS. After gentle pipetting a few times, the μ-bundles were centrifuged at 4000×g for 2 minutes and resuspended in 100 μL fresh PBS. Collagen μ-bundle suspension can be incorporated into another hydrogel or mixed with cells to create a 3D tissue model with complex ECM architectures.

### Fabrication of Mesoscale Collagen Strips Via Molecular Crowding

A series of cylindrical microchannels were molded in silicone polymer polydimethylsiloxane (PDMS) with needles (500 μm in diameter) (FIG. 2B). The channel device was sterilized with 70% ethanol and rinsed with sterile PBS before use. Once the channels were air-dried, they were filled with the PEG bath, and a collagen solution was gently injected into the PEG-filled channels. The collagen at the collagen-PEG interface rapidly gelled by the crowding effect while the collagen solution continued filling in the channel (FIG. 2C). After most of the PEG was replaced by the collagen, the collagen was allowed to stay in the channel for two more minutes for complete gelation. The collagen gel formed a cylindrical strip against the channel in the surrounding PEG at the collagen-channel interface. This small amount of remaining PEG prevented the collagen from adhering to the channel surface. Thus, the collagen strip was carefully washed out of the channel without any damage. Cells at varied densities can be mixed in collagen and fabricated into free-floating microtissues.

### Fabrication of Mesoscale Collagen Disks Via Molecular Crowding

A simple way to make circular collagen gels (FIG. 2D) was demonstrated. A small collagen droplet (tested volume: 10˜50 μL) was formed at the tip of a micropipette. The tip was slowly lowered until the collagen droplet made contact with the flat surface of the PEG bath. The droplet instantly expanded and spread uniformly into a disk. The disk was forced to polymerize rapidly. This procedure can be repeated numerous times to render a large number of microdisks. These microdisks can be incorporated with cells and washed in PBS to build microtissues.

### Fabrication of Collagen-Based Microparticles

Using the same principle for making the disks, collagen microdroplets were sprayed into a PEG bath by plunging a syringe filled with a small amount of collagen solution (FIG. 2E). When falling onto the PEG bath, these droplets (typically a few tens of micrometers in diameter) were instantly polymerized into microparticles. Due to the expansion of the droplets on the PEG bath, these particles often exhibit oblate or disk shapes.

### Free-Hand Printing of Collagen on a Surface

Instant gelation of hand-drawn collagen patterns were demonstrated on a surface. First, a glass coverslip or a PDMS block was treated with oxygen plasma to render a hydrophilic surface. A collagen solution was then hand-drawn onto the surface with a fine needle or a 10-μL micropipette tip (FIG. 2F). These surfaces with designed collagen patterns were carefully transformed onto a PEG bath and rapidly plunged into the PEG bath. When submerged in PEG, the collagen patterns were instantly polymerized. The PEG bath was then replaced with fresh PBS. The residue PEG on the printed collagen patterns was washed away.

In Situ Derivation of the Intestinal Tubes from iPSC-Strips

On Day 0, ˜80% confluent human induced pluripotent stem cells (iPSCs) were dissociated into single cells with the Gentle Cell Dissociation Reagent (STEMCELL Technologies). Bioink consisting of 1.5 mg/mL collagen with 5% Geltrex and high density iPSCs (4×10⁷ cells/mL) was prepared by mixing equal volumes of 3 mg/mL neutralized collagen with 10% Geltrex and 8×10⁷ cells/mL cell suspension in mTeSR 1 (STEMCELL Technologies) containing 10 μM ROCK inhibitor Y27632 (Y27). The bioink was rapidly crowded in the multi-channel device with a 400 μm channel diameter to generate the mesoscale tissue strips. These iPSC strips were subsequently rinsed in a PBS bath and then maintained at 37° C. in mTeSR 1 with Y27 for several hours. Individual strips with small amount of medium were then carefully moved to pre-chilled micro-centrifuge tubes and then mixed with cold reduced growth factor basement membrane matrix Geltrex (Thermo Fisher). The dilution of Geltrex by the introduced medium was less than 20%. A wide bore pipet tip was used to pick up ˜100 μL Geltrex solution with a single iPSC strip and slowly lay out the strip in an elongated gel dome onto a petri dish surface. After 20 min gelation at RT, the iPSC strips in the Geltrex were covered with Y27 containing mTeSR and cultured overnight. On Day 1, the Geltrex domes with the iPSC strips were gently detached from the petri dish and cultured in fresh mTeSR without Y27 for 6 hours to promote cell self-assembly. Once the strips showed smooth boundary, as an indication of proper cell merging and polarity, the intestinal organoid differentiation on the strips was initiated using a commercially available kit STEMdiff™ Intestinal Organoid Kit (05140, STEMCELL Technologies) containing necessary medium for each differentiation stage. The manufacturer's technical manual was strictly followed to prepare the inductions media. With the DE induction medium, the hPSC strips were first differentiated into DE strips for 3 days with daily medium change. To guarantee a full encapsulation in the basement membrane niche, the floating DE strips were embedded once again in high density Geltrex that was anchored on a 6-well plate. With the mid-hind gut induction medium, these embedded DE strips were then induced into mid-hind gut strips for 5 days with daily medium change. In the intestinal Organoid Growth Medium (OGM) with the OGM supplement and 1% GlutaMax (Thermo Fisher), the mid-hind gut strips were allowed to develop and mature in the Geltrex capsule for up to 5 weeks into intestinal specific tubes.

### Fabrication of Mouse Intestinal Tubes

Mouse intestinal organoids were collected before experiments. 1 mL gentle cell dissociation (STEMCELL Technology) was added to each well. The solution was pipetted up and down to break the Matrigel dome, twenty domes were collected and centrifuged at 4° C., 200×g for 5 minutes. Supernatant was discarded and 10 mL, 4° C. DMEM/F12 was added to resuspend the pellet. The suspension was centrifuged again at 4° C., 200×g for 5 minutes and supernatant was discarded. The pellet was then resuspended in 1 mL of neutralized 2 mg/mL collagen, and centrifuged again at 4° C., 200×g for 5 minutes to enrich the broken organoids at the bottom. The excessive collagen solution was removed. The PDMS channels were filled with 200 mg/mL PEG 8000, and 6 μL concentrated organoid bioink was pipetted into the channel. After 2 minutes, intestinal strips were washed out by sterile PBS. Then the strips were embedded in Matrigel and cultured in 6 well tissue culture plates for 5 days. Three (3) mL Intesticult medium (STEMCELL Technology) was added to each well and the medium was changed every 2 days.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of forming crowded collagen based constructs, comprising:
providing a collagen solution in a buffer;
providing a macromolecular bath solution; and
delivering the collagen solution into the macromolecular bath solution to form crowded collagen constructs.

2. The method of claim 1, wherein the macromolecular bath solution comprises a high molecular weight polyethylene glycol (PEG), wherein the molecular weight of the PEG is ranging between 0-30000 Da.

3. The method of claim 2, wherein the concentration of the PEG in the macromolecular bath solution is ranging between 0-1000 mg/mL.

4. The method of claim 1, wherein the buffer is selected from the group consisting of: NaOH, phosphate buffered saline (PBS), cell culture media, acetic acid, hydrochloric acid, pepsin and water.

5. The method of claim 1, wherein the collagen solution comprises collagen type I.

6. The method of claim 1, wherein the collagen solution is neutralized and has a collagen concentration ranging between 0-30 mg/mL.

7. The method of claim 1, wherein the collagen solution is acid solubilized and has a collagen concentration ranging between 0-30 mg/mL.

8. The method of claim 1, further comprising the step of adding one or more cells to the collagen solution; wherein the one or more cells are selected from the group consisting of: endothelial cells, stem cells, induced pluripotent stem cells, cancer cells, stem cell-derived cells, stromal cells, fibroblasts, immune cells, somatic cells, blood cells, and reproductive cells.

9. The method of claim 1, further comprising the step of adding one or more materials to the collagen solution; wherein the one or more materials are selected from the group consisting of: extracellular matrix proteins, proteoglycans, fibrinogen, thrombin, fibrin, collagen IV, laminin, basement membrane proteins, Matrigel, and Geltrex.

10. The method of claim 1, further comprising the step of adding one or more materials to the macromolecular bath solution; wherein the one or more materials are selected from the group consisting of: agarose, gelatin, slurry material, sacrificial slurry material, methylcellulose, and viscosity-modifying reagent.

11. The method of claim 1, wherein the step of delivering the collagen solution into the macromolecular bath solution comprises using any of a pipette, a syringe, a nozzle, to extrude the collagen solution into the macromolecular bath solution.

12. The method of claim 1, further comprising: providing a mold, filling the mold with the macromolecular bath solution, and depositing the collagen solution into the mold.

13. The method of claim 12, wherein the step of depositing the collagen solution into the mold comprises flowing the collagen solution into the mold.

14. The method of claim 1, wherein the step of delivering the collagen solution into the macromolecular bath solution comprises aerosolizing the collagen solution and spraying the collagen solution into the macromolecular bath solution.

15. A crowded collagen construct formed by the method of claim 1, wherein the construct formed comprises at least one of a disk, an elongated strip, a vascularized tissue construct, a microvascular network construct, a cell-laden construct, a patterned construct, and a fiber-like bundle.

16. The crowded collagen construct of claim 15, wherein the fiber-like bundles have diameters ranging from 0-1000 μm.

17. The crowded collagen construct of claim 15, wherein the fiber-like bundles have diameters greater than 1000 μm.

18. The crowded collagen construct of claim 15, wherein the fiber-like bundles are formed into a porous scaffold.

19. The crowded collagen construct of claim 18, wherein the pore sizes of the scaffold range between 0-1000 μm.

20. The crowded collagen construct of claim 15, wherein the fiber-like bundles are formed into at least one of organoids, tumor spheroids, vasculature, and micro-vasculature.

21. The method of claim 1, further comprising any of differentiating the construct in situ, culturing the construct, maturing the construct, biochemically stimulating the construct, biophysically stimulating the construct, biologically stimulating the construct, biochemically modifying the construct, biophysically modifying the construct, and biologically modifying the construct.

22. A method of forming crowded cellular based constructs, comprising:
providing a cellular solution in a buffer;
providing a macromolecular bath solution; and
delivering the cellular solution into the macromolecular bath solution to form crowded cellular constructs.

23. The method of claim 22, wherein the macromolecular bath solution comprises a high molecular weight polyethylene glycol (PEG), wherein the molecular weight of the PEG is ranging between 0-30000 Da.

24. The method of claim 23, wherein the concentration of the PEG in the macromolecular bath solution is ranging between 0-1000 mg/mL.

25. The method of claim 22, wherein the buffer is selected from the group consisting of: NaOH, phosphate buffered saline (PBS), cell culture media, acetic acid, hydrochloric acid, pepsin and water.

26. The method of claim 22, further comprising the step of adding one or more cells to the cellular solution; wherein the one or more cells are selected from the group consisting of: parenchymal cells, collenchyma cells, sclerenchyma cells, xylem cells, phloem cells, meristematic cells, epidermal cells, chloroplasts, plastids, leucoplasts, chromoplasts, plasmodesma, amyloplast, guard cells, vessel element cells, and sieve tube element cells.

27. The method of claim 22, further comprising the step of adding one or more materials to the macromolecular bath solution; wherein the one or more materials are selected from the group consisting of: agarose, gelatin, slurry material, sacrificial slurry material, methylcellulose, and viscosity-modifying reagent.

28. The method of claim 22, wherein the step of delivering the cellular solution into the macromolecular bath solution comprises using any of a pipette, a syringe, a nozzle, to extrude the cellular solution into the macromolecular bath solution.

29. The method of claim 22, further comprising: providing a mold, filling the mold with the macromolecular bath solution, depositing the cellular solution into the mold.

30. The method of claim 29, wherein the step of depositing the cellular solution into the mold comprises flowing the cellular solution into the mold.

31. The method of claim 22, wherein the step of delivering the cellular solution into the macromolecular bath solution comprises aerosolizing the cellular solution and spraying the cellular solution into the macromolecular bath solution.

32. A crowded cellular construct formed by the method of claim 22, wherein the construct formed comprises at least one of a disk, an elongated strip, a vascularized tissue construct, a microvascular network construct, a cell-laden construct, a patterned construct, and a fiber-like bundle.
